SEQUENCE LISTING (171 sequences)

SEQ ID NO: 1 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWKNDLGALENYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 2 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaaaatgatttgggcgcgctcgagaattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 3 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQYIQAIQAAKAAGMQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLGHVRGQTGKNLFAVGEFWKNDLGALENYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPSDGVPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 4 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaggccacgttcgaggtcaaacaggtaaaaacctttttgccgttggggaattttggaaaaatgatttgggcgcgctcgagaattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgagcgatggcgtcccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 5 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQYIQAIQAAKAAGMQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWKNDLGALENYITKTNGSMSLFDAPLHNNFYTASKSSGYFDMRNLLNGTLVQRHPSHAVTLVDNHDTQPGEALESFVQGWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 6 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaaaatgatttgggcgcgctcgagaattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacacaaccgggagaagcgctggaatcatttgttcaaggatggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 7 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQYIQAIQAAKAAGMQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWKNDLGALENYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPSDGVPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 8 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaaaatgatttgggcgcgctcgagaattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgagcgatggcgtcccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 9 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQLERAIRSLKANGIQVYADVVFNHKAGADQTEFVDAVEVDPSNRNQETSGTYQIQAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRNLLNGTLVQRHPSHAVTLVDNHDTQPGEALESFVQGWFKPLAYAFILTRQEGYPCVFYGDYYGIPSDGVPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 10 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcaggagcggaccaaacagaatttgtcgatgcggttgaggtagacccttctaatcgaaatcaagaaacatctggaacatatcaaattcaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacacaaccgggagaagcgctggaatcatttgttcaaggatggtttaaaccacttgcttacgcctttattttaacgagacaagagggatatccttgcgtattttacggtgactattatggaatcccgagcgatggcgtcccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 11 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQLERAIRSLKANGIQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRNLLNGTLVQRHPSHAVTLVDNHDTQPGEALESFVQGWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 12 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacacaaccgggagaagcgctggaatcatttgttcaaggatggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 13 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQLERAIRSLKANGIQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPSDGVPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 14 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgagcgatggcgtcccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 15 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQYIQAIQAAKAAGMQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPSDGVPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 16 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgagcgatggcgtcccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 17 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQLERAIRSLKANGIQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPSDGVPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 18 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgagcgatggcgtcccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 19 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQYIQAIQAAKAAGMQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRYLLNNTLMKDQPSLAVTLVDNHDTQPGEALESFVQGWFKPLAYAFILTRQEGYPCVFYGDYYGIPSDGVPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 20 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacacaaccgggagaagcgctggaatcatttgttcaaggatggtttaaaccacttgcttacgcctttattttaacgagacaagagggatatccttgcgtattttacggtgactattatggaatcccgagcgatggcgtcccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 21 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWKNDLGALENYITKTNGSMSLFDAPLHNNFYTASKSSGYFDMRNLLNGTLVQRHPSHAVTLVDNHDTQPGEALESFVQGWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 22 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaaaatgatttgggcgcgctcgagaattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacacaaccgggagaagcgctggaatcatttgttcaaggatggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 23 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQYIQAIQAAKAAGMQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKYSFFPDWLTYVRNQTGKNLFAVGEFWKNDLGALENYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 24 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatacagctttttccctgactggctaacatatgtacgtaatcaaacaggaaaaaatttatttgccgttggggaattttggaaaaatgatttgggcgcgctcgagaattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 25 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQYIQAIQAAKAAGMQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 26 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 27 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 28 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 29 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRNLLNGTLVQRHPSHAVTLVDNHDTQPGEALESFVQGWFKPLAYAFILTRQEGYPCVFYGDYYGIPSDGVPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 30 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacacaaccgggagaagcgctggaatcatttgttcaaggatggtttaaaccacttgcttacgcctttattttaacgagacaagagggatatccttgcgtattttacggtgactattatggaatcccgagcgatggcgtcccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 31 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQLERAIRSLKANGIQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLGHVRGQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPSDGVPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 32 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaggccacgttcgaggtcaaacaggtaaaaacctttttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgagcgatggcgtcccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 33 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQLERAIRSLKANGIQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 34 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggtaaaaacctttttgccgttggggaattttggagctatgacgtcaataagctgcataattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 35 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQYIQAIQAAKAAGMQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 36 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 37 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQLERAIRSLKANGIQVYADVVFNHKAGADGTEFVDAVEVDPSNRNQETSGTYQIQAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 38 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaaacatctggaacatatcaaattcaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 39 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 40 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 41 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQLERAIRSLKANGIQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRNLLNGTLVQRHPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 42 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgtaatttgctgaacggaacgcttgtccaacgacacccttcactcgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 43 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQYIQAIQAAKAAGMQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRYLLNNTLMKDQPSLAVTLVDNHDTQPGEALESFVQGWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 44 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacacaaccgggagaagcgctggaatcatttgttcaaggatggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 45 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRYLLNNTLMKDQPSLAVTLVDNHDTQPGEALESFVQGWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 46 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacacaaccgggagaagcgctggaatcatttgttcaaggatggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 47 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKYSFFPDWLGHVRGQTGKNLFAVGEFWSYDVNKLHNYITKTNGSMSLFDAPLHNNFYTASKSSGYFDMRYLLNNTLMKDQPSLAVTLVDNHDTQPGEALESFVQGWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 48 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatacagctttttccctgactggctaggccacgttcgaggtcaaacaggtaaaaacctttttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacacaaccgggagaagcgctggaatcatttgttcaaggatggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 49 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRNLLNGTLVQRHPSHAVTLVDNHDTQPGEALESFVQGWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 50 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacacaaccgggagaagcgctggaatcatttgttcaaggatggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 51 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWKNDLGALENYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 52 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaaaatgatttgggcgcgctcgagaattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 53 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQYIQAIQAAKAAGMQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPSDGVPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 54 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgagcgatggcgtcccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 55 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQYIQAIQAAKAAGMQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGEALESFVQGWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 56 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatacagctttttccctgactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacacaaccgggagaagcgctggaatcatttgttcaaggatggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 57 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPSDGVPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 58 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgagcgatggcgtcccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 59 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPSDGVPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 60 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgagcgatggcgtcccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 61 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPSDGVPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 62 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgagcgatggcgtcccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 63 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLGHVRGQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 64 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaggccacgttcgaggtcaaacaggtaaaaacctttttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 65 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKYSFFPDWLTYVRNQTGKNLFAVGEFWKNDLGALENYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 66 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatacagctttttccctgactggctaacatatgtacgtaatcaaacaggaaaaaatttatttgccgttggggaattttggaaaaatgatttgggcgcgctcgagaattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 67 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRYLLNNTLMKDQPSLAVTLVDNHDTQPGEALESFVQGWFKPLAYAFILTRQEGYPCVFYGDYYGIPSDGVPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 68 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacacaaccgggagaagcgctggaatcatttgttcaaggatggtttaaaccacttgcttacgcctttattttaacgagacaagagggatatccttgcgtattttacggtgactattatggaatcccgagcgatggcgtcccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 69 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLGHVRGQTGKNLFAVGEFWSYDVNKLHNYITKTNGSMSLFDAPLHNNFYTASKSSGYFDMRYLLNNTLMKDQPSLAVTLVDNHDTQPGEALESFVQGWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 70 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaggccacgttcgaggtcaaacaggtaaaaacctttttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacacaaccgggagaagcgctggaatcatttgttcaaggatggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 71 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQYIQAIQAAKAAGMQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 72 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 73 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQYIQAIQAAKAAGMQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 74 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 75 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 76 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 77 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQYIQAIQAAKAAGMQVYADVVFNHKAGADQTEFVDAVEVDPSNRNQETSGTYQIQAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 78 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcaggagcggaccaaacagaatttgtcgatgcggttgaggtagacccttctaatcgaaatcaagaaacatctggaacatatcaaattcaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 79 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 80 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 81 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 82 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 83 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRYLLNGTLVQRHPSHAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 84 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 85 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRYLLNNTLMKDQPSLAVTLVDNHDTQPGEALESFVQGWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 86 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacacaaccgggagaagcgctggaatcatttgttcaaggatggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 87 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQLERAIRSLKANGIQVYADVVFNHKAGADGTEFVDAVEVDPSNRNQETSGTYQIQAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 88 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaaacatctggaacatatcaaattcaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 89 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPSDGVPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 90 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgagcgatggcgtcccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 91 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADQTEFVDAVEVDPSNRNQETSGTYQIQAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRYLLNNTLMKDQPSLAVTLVDNHDTQPGEALESFVQGWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 92 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcaggagcggaccaaacagaatttgtcgatgcggttgaggtagacccttctaatcgaaatcaagaaacatctggaacatatcaaattcaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacacaaccgggagaagcgctggaatcatttgttcaaggatggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 93 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPSDGVPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 94 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgagcgatggcgtcccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 95 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 96 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcaggagcggaccaaacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 97 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 98 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 99 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 100 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatacagctttttccctgactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 101 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 102 - DNA, 1449 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcgggagctgacggcacagaatttgtcgatgcggttgaggtagacccttctaatcgaaatcaagaaacatctggaacatatcaaattcaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatacagctttttccctgactggctaacatatgtacgtaatcaaacaggaaaaaatttatttgccgttggggaattttggagctatgacgtcaataagctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaataa

SEQ ID NO: 103 - protein, 76 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIR

SEQ ID NO: 104 - protein, 76 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIR

SEQ ID NO: 105 - protein, 132 aa, Artificial Sequence
  Synthetically generated
TKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRGKAWDWEVDTENGNYDYLMFADLDMDHP

SEQ ID NO: 106 - protein, 132 aa, Artificial Sequence
  Synthetically generated
TKYGTKTQYIQAIQAAKAAGMQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHP

SEQ ID NO: 107 - protein, 132 aa, Artificial Sequence
  Synthetically generated
TKYGTKTQYIQAIQAAKAAGMQVYADVVFNHKAGADQTEFVDAVEVDPSNRNQETSGTYQIQAWTKFDFPGRGNTYSSFKWRWYHFDGTDWDESRKLNRIYKFRGKAWDWEVDTENGNYDYLMFADLDMDHP

SEQ ID NO: 108 - protein, 132 aa, Artificial Sequence
  Synthetically generated
TKYGTKTQYIQAIQAAKAAGMQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHP

SEQ ID NO: 109 - protein, 132 aa, Artificial Sequence
  Synthetically generated
TKYGTKTQLERAIRSLKANGIQVYADVVFNHKAGADGTEFVDAVEVDPSNRNQETSGTYQIQAWTKFDFPGRGNTYSSFKWRWYHFDGTDWDESRKLNRIYKFRGKAWDWEVDTENGNYDYLMFADLDMDHP

SEQ ID NO: 110 - protein, 132 aa, Artificial Sequence
  Synthetically generated
TKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHP

SEQ ID NO: 111 - protein, 132 aa, Artificial Sequence
  Synthetically generated
TKYGTKTQLERAIRSLKANGIQVYADVVFNHKAGADQTEFVDAVEVDPSNRNQETSGTYQIQAWTKFDFPGRGNTYSSFKWRWYHFDGTDWDESRKLNRIYKFRGKAWDWEVDTENGNYDYLMFADLDMDHP

SEQ ID NO: 112 - protein, 132 aa, Artificial Sequence
  Synthetically generated
TKYGTKTQLERAIRSLKANGIQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHP

SEQ ID NO: 113 - protein, 92 aa, Artificial Sequence
  Synthetically generated
EVVTELKNWGTWYVNTTNIDGFRLDAVKHIKYSFFPDWLTYVRNQTGKNLFAVGEFWSYDVNKLHNYITKTNGSMSLFDAPLHNNFYTASKS

SEQ ID NO: 114 - protein, 92 aa, Artificial Sequence
  Synthetically generated
EVVTELKNWGTWYVNTTNIDGFRLDAVKHIKYSFFPDWLTYVRNQTGKNLFAVGEFWKNDLGALENYITKTNGSMSLFDAPLHNNFYTASKS

SEQ ID NO: 115 - protein, 92 aa, Artificial Sequence
  Synthetically generated
EVVTELKNWGTWYVNTTNIDGFRLDAVKHIKYSFFPDWLGHVRGQTGKNLFAVGEFWSYDVNKLHNYITKTNGSMSLFDAPLHNNFYTASKS

SEQ ID NO: 116 - protein, 92 aa, Artificial Sequence
  Synthetically generated
EVVTELKNWGTWYVNTTNIDGFRLDAVKHIKYSFFPDWLGHVRGQTGKNLFAVGEFWKNDLGALENYITKTNGSMSLFDAPLHNNFYTASKS

SEQ ID NO: 117 - protein, 92 aa, Artificial Sequence
  Synthetically generated
EVVTELKNWGTWYVNTTNIDGFRLDAVKHIKFSFMRDWLTYVRNQTGKNLFAVGEFWSYDVNKLHNYITKTNGSMSLFDAPLHNNFYTASKS

SEQ ID NO: 118 - protein, 92 aa, Artificial Sequence
  Synthetically generated
EVVTELKNWGTWYVNTTNIDGFRLDAVKHIKFSFMRDWLTYVRNQTGKNLFAVGEFWKNDLGALENYITKTNGSMSLFDAPLHNNFYTASKS

SEQ ID NO: 119 - protein, 92 aa, Artificial Sequence
  Synthetically generated
EVVTELKNWGTWYVNTTNIDGFRLDAVKHIKFSFMRDWLGHVRGQTGKNLFAVGEFWSYDVNKLHNYITKTNGSMSLFDAPLHNNFYTASKS

SEQ ID NO: 120 - protein, 92 aa, Artificial Sequence
  Synthetically generated
EVVTELKNWGTWYVNTTNIDGFRLDAVKHIKFSFMRDWLGHVRGQTGKNLFAVGEFWKNDLGALENYITKTNGSMSLFDAPLHNNFYTASKS

SEQ ID NO: 121 - protein, 182 aa, Artificial Sequence
  Synthetically generated
SGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 122 - protein, 182 aa, Artificial Sequence
  Synthetically generated
SGYFDMRYLLNNTLMKDQPSLAVTLVDNHDTQPGQSLQSWVEPWFKPLAYAFILTRQEGYPCVFYGDYYGIPSDGVPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 123 - protein, 182 aa, Artificial Sequence
  Synthetically generated
SGYFDMRYLLNNTLMKDQPSLAVTLVDNHDTQPGEALESFVQGWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 124 - protein, 182 aa, Artificial Sequence
  Synthetically generated
SGYFDMRYLLNNTLMKDQPSLAVTLVDNHDTQPGEALESFVQGWFKPLAYAFILTRQEGYPCVFYGDYYGIPSDGVPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 125 - protein, 182 aa, Artificial Sequence
  Synthetically generated
SGYFDMRNLLNGTLVQRHPSHAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 126 - protein, 182 aa, Artificial Sequence
  Synthetically generated
SGYFDMRNLLNGTLVQRHPSHAVTLVDNHDTQPGQSLQSWVEPWFKPLAYAFILTRQEGYPCVFYGDYYGIPSDGVPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 127 - protein, 182 aa, Artificial Sequence
  Synthetically generated
SGYFDMRNLLNGTLVQRHPSHAVTLVDNHDTQPGEALESFVQGWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 128 - protein, 182 aa, Artificial Sequence
  Synthetically generated
SGYFDMRNLLNGTLVQRHPSHAVTLVDNHDTQPGEALESFVQGWFKPLAYAFILTRQEGYPCVFYGDYYGIPSDGVPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 129 - protein, 182 aa, Artificial Sequence
  Synthetically generated
SGYFDMRNLLNGTLVQRHPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 130 - protein, 182 aa, Artificial Sequence
  Synthetically generated
SGYFDMRYLLNGTLVQRHPSHAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 131 - protein, 482 aa, Artificial Sequence
  Synthetically generated
HDGTNGTIMQYFEWNVPNDGQHWNRLHNNAQNLKNAGITAIWIPPAWKGTSQSDTGYGAYDLYDLGEFNQRGTVRTKYGTKAELERAIRSLKANGIQVYGDVVMNHKAGADQTEQVQAVEVNPQNRNQEVSGTYQIEAWTGFNFPGRGNQHSSFKWRWYHFDGTDFDQSRGLSNRIYKFRTKAWDWEVDTEFGNYDYLMYADLDMDHPEVINELNRWGVWYANTLNLDGFRLDAVKHIKFSFMRDWLGHVRGQTGKNLFAVAEYWKNDLGALENYLSKTNWTMSAFDVPLHYNLYQASNSSGNYDMRNLLNGTLVQRHPSHAVTFVDNHDTQPGEALESFVQGWFKPLAYATILTREQGYPQVFYGDYYGIPSDGVPSYRQQIDPLLAARQQYAYGTQHDYLDNQDVIGWTREGDSAHAGSGLATVMSDGPGGSKTMYVGTAHAGQVFKDITGNRTDTVTINSAGNGTFRCNKGSVSIWVKQ

SEQ ID NO: 132 - protein, 583 aa, Artificial Sequence
  Bacillus sp.
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAKTSNVTFTVNNATTTSGQNVYVVANIPELGNWNTANAIKMNPSSYPTWKATIALPQGKAIEFKFIKKDQAGNVIWESTSNRTYTVPFSSTGSYTASWNVP

SEQ ID NO: 133 - protein, 484 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 134 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWKNDLGALENYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKTMYVGKKHAGKVFYDLTGNRSDTVTINADGNGTFKVNGGSVSIWVAK

SEQ ID NO: 135 - DNA, 1446 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaaaatgatttgggcgcgctcgagaattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaaacgatgtatgtcggtaaaaaacatgctggaaaagtattttatgatttaaccggaaaccgaagtgacacagtaacgattaatgcggatggtaacggaacgtttaaagtaaacggaggctccgtttcgatttgggtggctaaa

SEQ ID NO: 136 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWKNDLGALENYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKYMYVGKKHAGKVFYDLTGNRSDTVTINADGNGEFKVNGGSVSIWVAK

SEQ ID NO: 137 - DNA, 1446 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaaaatgatttgggcgcgctcgagaattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatatatgtatgtcggtaaaaaacatgctggaaaagtattttatgatttaaccggaaaccgaagtgacacagtaacgattaatgcggatggtaacggagaatttaaagtaaacggaggctccgtttcgatttgggtggctaaa

SEQ ID NO: 138 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWKNDLGALENYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKRMYVGKKHAGKVFYDLTGNRSDTVTINADGNGTFKVNGGSVSIWVAK

SEQ ID NO: 139 - DNA, 1446 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaaaatgatttgggcgcgctcgagaattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaacgcatgtatgtcggtaaaaaacatgctggaaaagtattttatgatttaaccggaaaccgaagtgacacagtaacgattaatgcggatggtaacggaacgtttaaagtaaacggaggctccgtttcgatttgggtggctaaa

SEQ ID NO: 140 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWKNDLGALENYITKTNGSMSLFDAPLHNNFYTASKSSGYFDMRNLLNGTLVQRHPSHAVTLVDNHDTQPGQSLQSWVEEWFKPLAYAFILTRQEGYPCVFYGDYYGIPKYNIPGMKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 141 - DNA, 1446 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaaaatgatttgggcgcgctcgagaattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaagaatggtttaaaccacttgcttacgcctttattttaacgagacaagagggatatccttgcgtattttacggtgactattatggaatcccgaaatacaatattccaggaatgaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaa

SEQ ID NO: 142 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWKNDLGALENYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPKHGIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 143 - DNA, 1446 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaaaatgatttgggcgcgctcgagaattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaacatggcattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaa

SEQ ID NO: 144 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWKNDLGALENYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPKYNIPGLKSKIDPLLEARRDYAYGTQRDYIDHQDIIGWTREGIDTKPNSGLAALITDGPGGSKWMYVGKKHAGKVFYDITGNRSDTVTINADGWGEFKVNGGSVSIWVAK

SEQ ID NO: 145 - DNA, 1446 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaaaatgatttgggcgcgctcgagaattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttagaagctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatatcaccggaaaccgaagtgacacagtaacgattaatgcggatggttggggagaatttaaagtaaacggaggctccgtttcgatttgggtggctaaa

SEQ ID NO: 146 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWKNDLGALENYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPKYNIPGLKSKIDPLLEARRDYAYGTQRDYIDHQDIIGWTREGIDTKPNSGLAALITDGPGGSKWMYVGKKHAGKVFKDITGNRSDTVTINADGWGEFKVNGGSVSIWVAQ

SEQ ID NO: 147 - DNA, 1446 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaaaatgatttgggcgcgctcgagaattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttagaagctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtatttaaagatatcaccggaaaccgaagtgacacagtaacgattaatgcggatggttggggagaatttaaagtaaacggaggctccgtttcgatttgggtggctcag

SEQ ID NO: 148 - protein, 482 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWKNDLGALENYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGDSSHPNSGLAALITDGPGGSKWMYVGKKHAGKVFYDLTGNRSDTVTINADGWGEFKVNGGSVSIWVAK

SEQ ID NO: 149 - DNA, 1446 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaaaatgatttgggcgcgctcgagaattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcgatagcagccatccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaa

SEQ ID NO: 150 - protein, 482 aa, Artificial Sequence
  Synthetically generated
NTAPTNETMMQYFEWDLPNDGTLWNRLHNNAQNLKNAGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWKNDLGALENYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPKYNIPGLKSKIDPLLIARQDYAYGTQRDYIDHQDIIGWTREGDSSHPNSGLAALITDGPGGSKWMYVGKKHAGKVFYDLTGNRSDTVTINADGWGEFKVNGGSVSIWVAK

SEQ ID NO: 151 - DNA, 1446 bp, Artificial Sequence
  Synthetically generated
aatactgcacctacgaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaaaatgatttgggcgcgctcgagaattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcaggactatgcctatggaacacaacgtgattacattgaccatcaagacattattggatggacacgcgaaggcgatagcagccatccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaa

SEQ ID NO: 152 - protein, 482 aa, Artificial Sequence
  Synthetically generated
NTAPTNETMMQYFEWDLPNDGTLWNRLHNNAQNLKNAGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWKNDLGALENYITKTNGSMSLFDAPLHNNFYTASKSSGYFDMRNLLNGTLVQRHPSHAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGDSSHPNSGLAALITDGPGGSKWMYVGKKHAGKVFYDLTGNRSDTVTINADGWGEFKVNGGSVSIWVAK

SEQ ID NO: 153 - DNA, 1446 bp, Artificial Sequence
  Synthetically generated
aatactgcacctacgaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaaaatgatttgggcgcgctcgagaattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcgatagcagccatccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaa

SEQ ID NO: 154 - protein, 482 aa, Artificial Sequence
  Synthetically generated
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKYSFFRDWLTYVRNQTGKNLFAVGEFWKYDVNALHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 155 - DNA, 1446 bp, Artificial Sequence
  Synthetically generated
aatactgcacctattaacgaaacaatgatgcaatattttgaatgggatttaccgaacgatggaaccctttggacaaaggtgaaaaatgaagccgcaaatctttcttcgctcggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaatatattcaagccatccaagctgccaaagccgcagggatgcaagtatatgcagatgttgtctttaatcataaggcgggagctgacggcacagaatttgtcgatgcggttgaggtagacccttctaatcgaaatcaagaaacatctggaacatatcaaattcaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatacagctttttccgcgactggctaacatatgtacgtaatcaaacaggaaaaaatttatttgccgttggggaattttggaaatatgacgtcaatgcgctgcataattacattacaaaaacaaatggatcgatgtcattatttgatgcacctttgcataacaacttttataccgcttccaaatcgagtggatattttgacatgcgttatttattgaataatacattaatgaaagatcaaccttcactcgctgtgacacttgtcgataaccacgacacgcaaccagggcaatctttacagtcatgggtcgaaccttggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaa

SEQ ID NO: 156 - protein, 483 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRALQNRIYKFRGKAWDWEVDTENGNYDYLMFADLDMDHPEVVTELQHWGTWYVNTTNIDGFRLDAVKHIKFSFMRDWLTYVRNQTGKNLFAVGEFWKNDLGALENYITKTNGSMSLFDAPLHENFYTASKSNGYFDMRDLLNGTLVQRHPSHAVTFVDNHDTDPEQSLDSWVEEWFKPLAYAFILTRQEGYPCVFYGDYYGIPKRNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 157 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgagcgttacagaatcggatttacaaattccgcggaaaagcatgggactgggaagtcgatacagaaaacggaaactatgattatttaatgttcgctgatttagatatggatcaccctgaggttgtgacagaattacagcattggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaaaatgatttgggcgcgctcgagaattacattacaaaaacaaatggatcgatgtcattatttgatgcacctttgcatgaaaacttttataccgcttccaaatcgaacggatattttgacatgcgtgatttgctgaacggaacgcttgtccaacgacacccttctcacgctgtgacatttgtcgataaccacgacacggatccagaacaatctttagattcatgggtcgaagaatggtttaaaccacttgcttacgcctttattttaacgagacaagagggatatccttgcgtattttacggtgactattatggaatcccgaaacgcaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaa

SEQ ID NO: 158 - protein, 482 aa, Artificial Sequence
  Synthetically generated
NTAPINETMMQYFEWDLPDDGTLWNRLHNNAQNLKNAGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRSLNRIYKFRGKAWDWEVDTENGNYDYLMFADLDMDHPEVVTELQNWGTWYVNTTNIDGFRLDAVKHIKFSFMRDWLTYVRNQTGKNLFAVGEFWKDDLGALENYITKTNGSMSLFDAPLHNNFYTASKSDGYFDMRNLLNGTLVERHPSHAVTLVDEHDTQPGESLQSWVEPWFKPLAYAFILTRQEGYPCVFYGDYYGIPKYDIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 159 - DNA, 1446 bp, Artificial Sequence
  Synthetically generated
aatactgcacctattaacgaaacaatgatgcaatattttgaatgggatttaccggatgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaagcttaaatcggatttacaaattccgcggaaaagcatgggactgggaagtcgatacagaaaacggaaactatgattatttaatgttcgctgatttagatatggatcaccctgaggttgtgacagaattacagaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaagatgatttgggcgcgctcgagaattacattacaaaaacaaatggatcgatgtcattatttgatgcacctttgcataacaacttttataccgcttccaaatcggatggatattttgacatgcgtaatttgctgaacggaacgcttgtcgaacgacacccttctcacgctgtgacacttgtcgatgaacacgacacgcaaccaggggaatctttacagtcatgggtcgaaccttggtttaaaccacttgcttacgcctttattttaacgagacaagagggatatccttgcgtattttacggtgactattatggaatcccgaaatacgatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaa

SEQ ID NO: 160 - protein, 483 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRTLQNRIYKFRGKAWDWEVDTENGNYDYLMFADLDMDHPEVVTELQKWGTWYVNTTNIDGFRLDAVKHIKFSFMRDWLTYVRNQTGKNLFAVGEFWKNDLGALENYITKTNGSMSLFDAPLHNEFYTASKSNGYFDMRELLEGTLVQRHPSHAVTYVDNHDTQPEQSLQSWVEEWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 161 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaacgttacagaatcggatttacaaattccgcggaaaagcatgggactgggaagtcgatacagaaaacggaaactatgattatttaatgttcgctgatttagatatggatcaccctgaggttgtgacagaattacagaaatggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaaaatgatttgggcgcgctcgagaattacattacaaaaacaaatggatcgatgtcattatttgatgcacctttgcataacgaattttataccgcttccaaatcgaacggatattttgacatgcgtgaattgctggaaggaacgcttgtccaacgacacccttctcacgctgtgacatatgtcgataaccacgacacgcaaccagaacaatctttacagtcatgggtcgaagaatggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaa

SEQ ID NO: 162 - protein, 483 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLQNRIYKFRGKAWDWEVDTENGNYDYLMFADLDMDHPEVVTELKNWGTWYVNTTNIDGFRLDAVKHIKFSFMRDWLTYVRNQTGKNLFAVGEFWKDDLGALENYITKTNGSMSLFDAPLHNNFYTASKSSGYFDMRNLLNGTLVQRHPSHAVTLVDNHDTDPGQSLESWVEPWFKPLAYAFILTRQEGYPCVFYGDYYGIPKYEIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 163 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattacagaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaagatgatttgggcgcgctcgagaattacattacaaaaacaaatggatcgatgtcattatttgatgcacctttgcataacaacttttataccgcttccaaatcgagtggatattttgacatgcgtaatttgctgaacggaacgcttgtccaacgacacccttctcacgctgtgacacttgtcgataaccacgacacggatccagggcaatctttagaatcatgggtcgaaccttggtttaaaccacttgcttacgcctttattttaacgagacaagagggatatccttgcgtattttacggtgactattatggaatcccgaaatacgaaattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaa

SEQ ID NO: 164 - protein, 482 aa, Artificial Sequence
  Synthetically generated
NTAPINETMMQYFEWDLPEDGTLWNRLHNNAQNLKNAGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWKEDLGALENYITKTNGSMSLFDAPLHENFYTASKSSGYFDMRELLNGTLVERHPSHAVTLVDEHDTQPGQSLESWVEPWFKPLAYAFILTRQEGYPCVFYGDYYGIPKYEIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 165 - DNA, 1446 bp, Artificial Sequence
  Synthetically generated
aatactgcacctattaacgaaacaatgatgcaatattttgaatgggatttaccggaagatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaaaactggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaagaagatttgggcgcgctcgagaattacattacaaaaacaaatggatcgatgtcattatttgatgcacctttgcatgaaaacttttataccgcttccaaatcgagtggatattttgacatgcgtgaattgctgaacggaacgcttgtcgaacgacacccttctcacgctgtgacacttgtcgatgaacacgacacgcaaccagggcaatctttagaatcatgggtcgaaccttggtttaaaccacttgcttacgcctttattttaacgagacaagagggatatccttgcgtattttacggtgactattatggaatcccgaaatacgaaattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaa

SEQ ID NO: 166 - protein, 483 aa, Artificial Sequence
  Synthetically generated
NTAPINETMMQYFEWDLPNDGTLWNRLHNNAQNLKNAGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRALQNRIYKFRGKAWDWEVDTENGNYDYLMFADLDMDHPEVVTELNRWGTWYVNTTNIDGFRLDAVKHIKFSFMRDWLTYVRNQTGKNLFAVGEFWKNDLGALENYITKTNGSMSLFDAPLHNNFYTASKSDGYFDMRNLLNGTLVQRHPSHAVTFVDNHDTQPGQSLQSWVEEWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 167 - DNA, 1449 bp, Artificial Sequence
  Synthetically generated
aatactgcacctattaacgaaacaatgatgcaatattttgaatgggatttaccgaacgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgagcgttacagaatcggatttacaaattccgcggaaaagcatgggactgggaagtcgatacagaaaacggaaactatgattatttaatgttcgctgatttagatatggatcaccctgaggttgtgacagaattaaaccgctggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaaaatgatttgggcgcgctcgagaattacattacaaaaacaaatggatcgatgtcattatttgatgcacctttgcataacaacttttataccgcttccaaatcggatggatattttgacatgcgtaatttgctgaacggaacgcttgtccaacgacacccttctcacgctgtgacatttgtcgataaccacgacacgcaaccagggcaatctttacagtcatgggtcgaagaatggtttaaaccacttgcttacgcctttattttaacgagacaagagggatatccttgcgtattttacggtgactattatggaatcccgaaatacaatattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaa

SEQ ID NO: 168 - protein, 482 aa, Artificial Sequence
  Synthetically generated
NTAPINETMMQYFEWDLPDDGTLWNRLHNNAQNLKNAGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELNRWGTWYVNTTNIDGFRLDAVKHIKFSFMRDWLTYVRNQTGKNLFAVGEFWKEDLGALENYITKTNGSMSLFDAPLHENFYTASKSSGYFDMRELLNGTLVQRHPSHAVTLVDEHDTQPGQSLESWVEPWFKPLAYAFILTRQEGYPCVFYGDYYGIPKYEIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 169 - DNA, 1446 bp, Artificial Sequence
  Synthetically generated
aatactgcacctattaacgaaacaatgatgcaatattttgaatgggatttaccggatgatggaaccctttggaatagactccataataatgcgcaaaaccttaagaatgcaggtattacagcgttatggcttcctccagcgtataaaggaacaagtcaaagcgatgtcggatacggcgtgtacgatttatatgaccttggggaatttaatcaaaaaggaacgattcgaacaaaatacggaacaaaaacacaactggaacgcgcaattagatccttaaaagctaacggaatccaagtatatgcagatgttgtctttaatcataaggcgggagctgacggcacagaatttgtcgatgcggttgaggtagacccttctaatcgaaatcaagaagtgagcggaacgtatcaaattgaagcatggacaaaatttgattttcccggtcgggggaacacatactcgagttttaaatggcgttggtatcattttgacggtaccgattgggatgaaagccgaaaattaaatcggatttacaaattccgcggaaaagcatgggactgggaagtcgatacagaaaacggaaactatgattatttaatgttcgctgatttagatatggatcaccctgaggttgtgacagaattaaaccgctggggaacgtggtacgtcaatactacaaatatcgatggattccgcttagatgccgtaaaacatattaaatttagttttatgcgggactggctaacatatgtacgtaatcaaacaggaaaaaatttatttgccgttggggaattttggaaagaagatttgggcgcgctcgagaattacattacaaaaacaaatggatcgatgtcattatttgatgcacctttgcatgaaaacttttataccgcttccaaatcgagtggatattttgacatgcgtgaattgctgaacggaacgcttgtccaacgacacccttctcacgctgtgacacttgtcgatgaacacgacacgcaaccagggcaatctttagaatcatgggtcgaaccttggtttaaaccacttgcttacgcctttattttaacgagacaagagggatatccttgcgtattttacggtgactattatggaatcccgaaatacgaaattccaggattaaaaagcaaaatcgacccgcttttaattgctcgtcgggactatgcctatggaacacaacgtgattacattgaccatcaagacattattggatggacacgcgaaggcattgatacaaaaccaaactctggactggcggctttaattaccgacggccctggcggaagcaaatggatgtatgtcggtaaaaaacatgctggaaaagtattttatgatttaaccggaaaccgaagtgacacagtaacgattaatgcggatggttggggagaatttaaagtaaacggaggctccgtttcgatttgggtggctaaa

SEQ ID NO: 170 - protein, 482 aa, Artificial Sequence
  Synthetically generated
HHNGTNGTMMQYFEWDLPNDGTLWNRLHNNAQNLKNAGITALWLPPAYKGTSQSDVGYGVYDLYDLGEFNQKGTIRTKYGTKTQYIQAIQAAKAAGMQVYADVVFNHKAGADQTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWSYDVNKLHNYITKTNGSMSLFDAPLHNNFYTASKSSGNFDMRNLLNGTLVQRHPSHAVTLVDNHDTQPGQSLQSWVEPWFKPLAYAFILTRQEGYPCVFYGDYYGIPSDGVPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 171 - protein, 482 aa, Artificial Sequence
  Synthetically generated
HHNGTNGTMMQYFEWDLPNDGTLWNRLHNNAQNLKNAGITALWLPPAYKGTSQSDVGYGVYDLYDLGEFNQKGTIRTKYGTKTQLERAIRSLKANGIQVYADVVFNHKAGADGTEFVDAVEVDPSNRNQEVSGTYQIEAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKFSFMRDWLTYVRNQTGKNLFAVGEFWKNDLGALENYITKTNGSMSLFDAPLHNNFYTASKSSGNFDMRNLLNGTLVQRHPSHAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVKGGSVSIWVAK

(19) United States
(12) Patent Application Publication
(10) Pub. No.: US 2022/0162576 A1
(43) Pub. Date: May 26, 2022

(54) AMYLASE ENZYMES

(71) Applicant: BASF SE, Ludwigshafen (DE)

(72) Inventors: Stefan Jenewein, Ludwigshafen (DE); Zachary David Miles, San Diego, CA (US); Asfia Qureshi, San Diego, CA (US); Jared Dennis, San Diego, CA (US); Tong Li, San Diego, CA (US); Xuqiu Tan, San Diego, CA (US); Priya Anand, Ludwigshafen (DE); Jesper Nielsen, San Diego, CA (US)

(21) Appl. No.: 17/442,489

(22) PCT Filed: Mar. 24, 2020
(86) PCT No.: PCT/EP2020/058130

§ 371 Date: Sep. 23, 2021

Related U.S. Application Data
(60) Provisional application No. 62/890,325, filed on Aug. 22, 2019.
(60) Provisional application No. 62/823,561, filed on Mar. 25, 2019.

(51) Int. Cl. C12N 9/28

(52) U.S. Cl. CPC C12N 9/2417; C12Y 302/01001

(57) ABSTRACT

Genetically engineered enzymes having amylase enzyme activity, compositions comprising the enzymes, and methods of making and using the enzymes. Fragments of parent amylase enzymes activity and methods of using the fragments for making genetically engineered enzymes having amylase activity. The genetically engineered amylase enzymes are useful in many different applications such as laundry detergents, dish washing detergents, and cleaning products for homes, industry, vehicle care, baking, animal feed, pulp and paper processing, starch processing, brewing, and ethanol production.

### SEQUENCE LISTING

This application includes a nucleotide and amino acid sequence listing in computer readable form (CRF) as an ASC II text (.txt) file according to “Standard for the Presentation of Nucleotide and Amino Acid Sequence Listings in International Patent Applications Under the Patent Cooperation Treaty (PCT)” ST.25. The sequence listing is identified below and is hereby incorporated by reference into the specification of this application in its entirety and for all purposes.

| File Name | Date of Creation | Size |
|---|---|---|
| Mar. 20, 2020 190332WO01 | Mar. 20, 2020 | 438 KB |
| Sequence Listing.txt | | |

### TECHNICAL FIELD

Genetically engineered amylase enzymes, compositions comprising the enzymes, and methods of using the enzymes or compositions comprising the enzymes. The genetically engineered amylase enzymes are useful in many different applications such as laundry detergents, dish washing detergents, and cleaning products for homes, industry, vehicle care, baking, animal feed, pulp and paper processing, starch processing, and ethanol production. Amylases have been employed in the removal of starch stains and have been added to various compositions such as cleaning products. Current cleaning and/or fabric care compositions comprise formulations of many active ingredients that impact with the ability of amylases to remove starch stains; however, many of these amylases do not retain activity especially at high temperatures such as 80 degrees C. or 90 degrees C. In addition, many of the enzymes do not retain activity in detergent formulations. These decreases in activity because of high temperatures or other formulations can negatively impact the amylase performance in washing. Thus, the need exists for genetically engineered amylase enzymes that can function in the harsh environment of compositions used for cleaning, high temperatures, and still have an effective performance of removing starch stains in a washing application.

### SUMMARY OF THE INVENTION

An amylase comprising an amino acid sequence that is at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171.

An amylase comprising an amino acid sequence that is at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, or SEQ ID NO:9.

An amylase comprising an amino acid sequence that is at least 95%, at least 96%, at least 97%, at least 98%, at least 98.5%, at least 99%, at least 99.5%, or 100% identical to the full length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, or SEQ ID NO:21.

An amylase comprising an amino acid sequence that is at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, or SEQ ID NO:43.

An amylase comprising an amino acid sequence that is at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, or SEQ ID NO:71.

An amylase comprising an amino acid sequence that is at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, or SEQ ID NO:91.

An amylase comprising an amino acid sequence that is at least 99% or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, or SEQ ID NO: 99.

An amylase comprising an amino acid sequence that is 99.5% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171.

An amylase comprising an amino acid sequence that is 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171.

In a preferred embodiment, the amylase comprises an amino acid sequence that is at least 94%, at least 95%, at least 96%, at least 97%, at least 98%, at least 98.5%, at least 99%, at least 99.5%, or 100% identical to the full-length amino acid sequence of: SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171.

In a further preferred embodiment, the amylase comprises an amino acid sequence that is at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:11, SEQ ID NO:15, SEQ ID NO:146, SEQ ID NO:148, SEQ ID NO: 152, SEQ ID NO: 154; SEQ ID NO: 170, or SEQ ID NO:171.

In a another preferred embodiment, the amylase comprises an amino acid sequence that is at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:15, SEQ ID NO:146, SEQ ID NO:148, SEQ ID NO: 152, SEQ ID NO: 154, SEQ ID NO: 170, or SEQ ID NO:171.

In a another preferred embodiment, the amylase comprises an amino acid sequence that is at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:15, SEQ ID NO:146, SEQ ID NO:148, SEQ ID NO: 152, SEQ ID NO: 170, or SEQ ID NO:171.

In another preferred embodiment, the amylase comprises an amino acid sequence that is at least 94%, at least 95%, at least 96%, at least 97%, at least 98%, at least 98.5%, at least 99%, at least 99.5%, or 100% identical to the full-length amino acid sequence of: SEQ ID NO:11. An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, and comprising a deletion of one or more, preferably two or three, most preferred two amino acid residues, at amino acid positions 180-183, preferably 180-182, according to the numbering of SEQ ID NO: 133.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, and comprising a deletion of one or more, preferably two or three, most preferred two amino acid residues, at amino acid positions 180-183, preferably 180-182, according to the numbering of SEQ ID NO: 133.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:11, SEQ ID NO:15, SEQ ID NO:146, SEQ ID NO:148, SEQ ID NO: 152, SEQ ID NO: 154; SEQ ID NO: 170, or SEQ ID NO:171, and comprising a deletion of one or more, preferably two or three, most preferred two amino acid residues, at amino acid positions 180-183, preferably 180-182, according to the numbering of SEQ ID NO: 133. An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, and comprising at least one, at least two, or at least three, most preferred two, deletions of amino acid residues selected from the group consisting of R180, S181, T182 and G183, preferably selected from the group consisting of R180, S181, and T182, according to the numbering of SEQ ID NO: 133.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:11, SEQ ID NO:15, SEQ ID NO:146, SEQ ID NO:148, SEQ ID NO: 152; SEQ ID NO: 170, or SEQ ID NO:171, and comprising at least one, at least two, or at least three, most preferred two, deletions of amino acid residues selected from the group consisting of R180, S181, T182 and G183, preferably selected from the group consisting of R180, S181, and T182, according to the numbering of SEQ ID NO: 133.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, and at least one polypeptide fragment selected from each of the following regions: Region 1, Region 2, Region 3, and Region 4. Herein, region 1 corresponds to the region of the amylase amino acid sequence defined by amino acid residues 1-76 according to the numbering of SEQ ID NO: 133; region 2 corresponds to the region of the amylase amino acid sequence defined by amino acid residues 77-210 according to the numbering of SEQ ID NO: 133; region 3 corresponds to the region of the amylase amino acid sequence defined by amino acid residues 211-302 according to the numbering of SEQ ID NO: 133, and region 4 corresponds to the region of the amylase amino acid sequence defined by amino acid residues 303-484 according to the numbering of SEQ ID NO: 133.

An amylase comprising at least one fragment of SEQ ID NO:101 and at least one fragment of SEQ ID NO:131, wherein the amino acid sequence of the at least one fragment of SEQ ID NO:101 differs from the corresponding fragment in SEQ ID NO:131 and wherein the amino acid sequence of the at least one fragment of SEQ ID NO:131 differs from the corresponding fragment in SEQ ID NO:101 and wherein the amylase comprises an amino acid sequence that is at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full length amino acid sequence of: SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, or SEQ ID NO:130.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, and at least one polypeptide fragment selected from each of the following regions: Region 1, Region 2, Region 3, and Region 4; wherein the polypeptide fragment from region 1 is an amino acid sequence having at least 75% identical, 76% identical, at least 77% identical, at least 78% identical, at least 79% identical, at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to an amino acid sequence selected from: SEQ ID NO:103, and SEQ ID NO:104.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, and at least one polypeptide fragment selected from each of the following regions: Region 1, Region 2, Region 3, and Region 4; wherein the polypeptide fragment from region 2 is an amino acid sequence having at least 75% identical, 76% identical, at least 77% identical, at least 78% identical, at least 79% identical, at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to an amino acid sequence selected from: SEQ ID NO:105, SEQ ID NO:106, SEQ ID NO:107, SEQ ID NO:108, SEQ ID NO:109, SEQ ID NO:110, SEQ ID NO:111, and SEQ ID NO:112.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, and at least one polypeptide fragment selected from each of the following regions: Region 1, Region 2, Region 3, and Region 4; wherein the polypeptide fragment from region 3 is an amino acid sequence having at least 75% identical, 76% identical, at least 77% identical, at least 78% identical, at least 79% identical, at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to an amino acid sequence selected from: SEQ ID NO:113, SEQ ID NO:114, SEQ ID NO:115, SEQ ID NO:116, SEQ ID NO:117, SEQ ID NO:118, SEQ ID NO:119, and SEQ ID NO:120.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, and at least one polypeptide fragment selected from each of the following regions: Region 1, Region 2, Region 3, and Region 4; wherein the polypeptide fragment from region 4 is an amino acid sequence is at least 75% identical, 76% identical, at least 77% identical, at least 78% identical, at least 79% identical, at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to an amino acid sequence selected from: SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, and SEQ ID NO:130.

An amylase comprising one or more amino acid residue insertions, deletions, substitutions, or any combinations thereof to the amino acid sequence of: SEQ ID NO: 1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171.

An amylase comprising one or more amino acid residue substitution to the amino acid sequence of: SEQ ID NO: 1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171.

An amylase wherein the amino acid sequence is encoded by a polynucleotide having a nucleic acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full length polynucleotide sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:68, SEQ ID NO:70, SEQ ID NO:72, SEQ ID NO:74, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, SEQ ID NO:161, SEQ ID NO:163, SEQ ID NO:165, SEQ ID NO:167, or SEQ ID NO:169.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or

SEQ ID NO:171, wherein the amylase has an increase in expression; activity; thermostability; stability; performance in laundry; or any combination thereof.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, wherein the increase in expression is from 1.01 to 1.27.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, wherein the increase in enzyme activity is from 12% to 121%.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, wherein the increase in enzyme activity is from 50% to 121%.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171; wherein the increase in thermostability is after a heat challenge at a temperature from 75 degrees C. to 95 degrees C.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, wherein the increase in thermostability is after a heat challenge at a temperature of: 80 degrees C., 81 degrees C., 82 degrees C., 83 degrees C., 84 degrees C., 85 degrees C., 86 degrees C., 87 degrees C., 88 degrees C., 89 degrees C., or 90 degrees C.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, wherein the increase in thermostability is after a heat challenge at a temperature of: 80 degrees C.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, wherein the increase in thermostability is after a heat challenge at a temperature of: 90 degrees C.

Preferably, the present invention is directed to an amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171; wherein the amylase has increased stability, preferably in a laundry detergent.

Preferably, the present invention is directed to an amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO: 134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171; wherein the amylase has increased stability, preferably in a laundry detergent.

Preferably, the present invention is directed to an amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:11, SEQ ID NO:15, SEQ ID NO:146, SEQ ID NO:148, SEQ ID NO: 152, SEQ ID NO: 154; SEQ ID NO: 170, or SEQ ID NO:171; wherein the amylase has increased stability, preferably in a laundry detergent.

Preferably, the present invention is directed to an amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:15, SEQ ID NO:146, SEQ ID NO:148, SEQ ID NO: 152, SEQ ID NO: 170, or SEQ ID NO:171; wherein the amylase has increased stability, preferably in a laundry detergent.

Preferably, the present invention is directed to an amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:15, SEQ ID NO:148, SEQ ID NO: 170, or SEQ ID NO:171; wherein the amylase has increased stability, preferably in a laundry detergent.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171; wherein the increase in stability is from 0.8% to 70%.

Preferably, an amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of SEQ ID NO: 134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171; wherein the increase in stability is from 0.8% to 70%.

Preferably, an amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of SEQ ID NO:1, SEQ ID NO:11, SEQ ID NO:15, SEQ ID NO:146, SEQ ID NO:148, SEQ ID NO: 152, SEQ ID NO: 154; SEQ ID NO: 170, or SEQ ID NO:171; wherein the increase in stability is from 0.8% to 70%.

Preferably, an amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of SEQ ID NO:1, SEQ ID NO:15, SEQ ID NO:146, SEQ ID NO:148, SEQ ID NO: 152, SEQ ID NO: 170, or SEQ ID NO:171; wherein the increase in stability is from 0.8% to 70%.

Preferably, an amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of SEQ ID NO:1, SEQ ID NO:15, SEQ ID NO:148, SEQ ID NO: 170, or SEQ ID NO:171; wherein the increase in stability is from 0.8% to 70%. An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, wherein the increase in stability is from 1.8% to 69.5%.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, wherein the increase in stability is from 1.7% to 49.3%.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, wherein the increase in stability is 0.8% to 21.6%.

In a preferred embodiment, the present invention is directed to an amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, wherein the amylase has increased in performance in laundry.

In a preferred embodiment, the present invention is directed to an amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO: 134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, wherein the amylase has increased in performance in laundry.

In a preferred embodiment, the present invention is directed to an amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:11, SEQ ID NO:15, SEQ ID NO:146, SEQ ID NO:148, SEQ ID NO: 152, SEQ ID NO: 154; SEQ ID NO: 170, or SEQ ID NO:171, wherein the amylase has increased in performance in laundry.

In a preferred embodiment, the present invention is directed to an amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:15, SEQ ID NO:146, SEQ ID NO:148, SEQ ID NO: 152, SEQ ID NO: 154; SEQ ID NO: 170, or SEQ ID NO:171, wherein the amylase has increased in performance in laundry.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, wherein the increase in performance in laundry is from 1.07 to 1.8 RGB.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO: 134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, wherein the increase in performance in laundry is from 1.07 to 1.8 RGB.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:11, SEQ ID NO:15, SEQ ID NO:146, SEQ ID NO:148, SEQ ID NO: 152, SEQ ID NO: 154; SEQ ID NO: 170, or SEQ ID NO:171, wherein the increase in performance in laundry is from 1.07 to 1.8 RGB.

An amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:15, SEQ ID NO:146, SEQ ID NO:148, SEQ ID NO: 152, SEQ ID NO: 154; SEQ ID NO: 170, or SEQ ID NO:171, wherein the increase in performance in laundry is from 1.07 to 1.8 RGB. A compositions comprising an amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171. In another embodiment, the composition further comprises a second enzyme selected from the group consisting of: a second amylase, a lipase, a protease, a cellulase, a laccase, a mannanase, a pectinase, a nuclease, and any combination thereof. In preferred embodiment, the composition further comprises a second enzyme and the second enzyme is a different amylase. In another preferred embodiment, the composition further comprises a second enzyme and the second enzyme is a protease.

A method of making the amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171; comprising: providing a nucleic acid sequence comprising: SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:68, SEQ ID NO:70, SEQ ID NO:72, SEQ ID NO:74, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, SEQ ID NO:161, SEQ ID NO:163, SEQ ID NO:165, SEQ ID NO:167, or SEQ ID NO:169; transforming the nucleic acid sequence into an expression host, cultivating the expression host to produce the amylase, and purifying the amylase.

The method of making the amylase wherein the expression host is selected from the group consisting of: a bacterial expression system, a yeast expression system, a fungal expression system, and a synthetic expression system.

The method of making the amylase wherein the bacterial expression system is selected from an E. coli, a Bacillus, a Pseudomonas, and a Streptomyces, wherein the yeast expression system is selected from a Candida, a Pichia, a Saccharomyces, a Schizosaccharomyces or, wherein the fungal expression system is selected from a Penicillium, an Aspergillus, a Fusarium, a Myceliopthora, a Themothelomyces, a Rhizomucor, a Rhizopus, a Thermomyces, and a Trichoderma.

A method of making the amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171; wherein the expression host is a Bacillus host cell.

A method of preparing a dough or a baked product prepared from the dough, the method comprising adding the amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, to the dough and baking it.

A method of use of the amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, for processing starch, for cleaning or washing textiles, hard surfaces, or dishes, for making ethanol, for processing pulp or paper, or for feeding an animal.

A method of use of the amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, for cleaning or washing textiles, hard surfaces, or dishes.

A method of use of the amylase comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to the full-length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171, for cleaning or washing textiles.

A method for making a hybrid amylase comprising: (a) providing an amylase having at least 80% identical to the full-length amino acid sequence of SEQ ID NO:101, SEQ ID NO:131, SEQ ID NO:132, or SEQ ID NO:133; (b) providing a polypeptide fragment having at least 75% sequence identity to an amino acid sequence selected from the amino acid sequence of: SEQ ID NO: 103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, and SEQ ID NO:130; (c) aligning the polypeptide fragments of (b) with the amino acid sequence of the amylase of (a) and identifying regions in the amylase of (a) that can be replaced by the fragments of (b);

(d) substituting the regions in the amylase of (a) with the fragments of (b) to generate the hybrid amylase.

In one embodiment, the amylase of the present invention is a variant amylase with an amino acid sequence comprising compared to the parent amylase at least one amino acid substitution, preferably, at least two, at least three, or at least four amino acid substitutions as described herein. The parent amylase sequence can be any amylase sequence described herein. Preferably, the parent sequence is an amino acid sequence shown in SEQ ID NO: 1-171, preferably, an amino acid sequence shown in SEQ ID NO: 1-102, more preferably the parent sequence is SEQ ID NO: 101, most preferably the parent sequence is SEQ ID NO: 133. In one embodiment, the amylase of the present invention is a non-naturally occurring amylase comprising an amino acid sequence having at least one amino acid substitution, preferably, at least two, at least three, or at least four amino acid substitutions as described herein. In one embodiment, the amylase of the present invention is a non-naturally occurring amylase comprising an amino acid sequence having at least one non-naturally occurring amino acid substitution, preferably, at least two, at least three, or at least four amino acid substitutions as described herein. Preferably, the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to an amino acid sequence shown in SEQ ID NO: 1-171 and comprises at least one amino acid substitution as described herein.

In one embodiment, the amylase of the present invention is a non-naturally occurring amylase comprising an amino acid sequence having at least one non-naturally occurring amino acid substitution, preferably, at least two, at least three, or at least four amino acid substitutions as described herein. Preferably, the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to an amino acid sequence shown in SEQ ID NO: 1-102 and comprises at least one amino acid substitution as described herein.

In one embodiment, the amylase of the present invention is a non-naturally occurring amylase comprising an amino acid sequence having at least one non-naturally occurring amino acid substitution, preferably, at least two, at least three, or at least four amino acid substitutions as described herein. Preferably, the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 101 and comprises at least one amino acid substitution as described herein.

In one embodiment, the amylase of the present invention is a non-naturally occurring amylase comprising an amino acid sequence having at least one non-naturally occurring amino acid substitution, preferably, at least two, at least three, or at least four amino acid substitutions as described herein. Preferably, the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 133 and comprises at least one amino acid substitution as described herein.

In one embodiment, the at least one amino acid substitution is in at least one of the regions of the amylase selected from the group consisting of region 1, region 2, region 3, and region 4, wherein region 1 corresponds to the region of the amylase amino acid sequence defined by amino acid residues 1-76 according to the numbering of SEQ ID NO: 133, wherein region 2 corresponds to the region of the amylase amino acid sequence defined by amino acid residues 77-210 according to the numbering of SEQ ID NO: 133, wherein region 3 corresponds to the region of the amylase amino acid sequence defined by amino acid residues 211-302 according to the numbering of SEQ ID NO: 133, and wherein region 4 corresponds to the region of the amylase amino acid sequence defined by amino acid residues 303-484 according to the numbering of SEQ ID NO: 133.

Preferably, the at least one amino acid substitution is in at least one subregion of at least one region of the amylase selected from the group of regions consisting of region 1, region 2, region 3, and region 4. Preferably, the subregion of region 1 of the amino acid sequence of the amylase is defined by amino acid residues 25-37 according to the numbering of SEQ ID NO: 133. Preferably, the subregion of region 2 of the amino acid sequence of the amylase is selected from the group consisting of the subregions that are defined by amino acid residues 85-97, 113, and 131-138 according to the numbering of SEQ ID NO: 133. Preferably, the subregion of region 3 of the amino acid sequence of the amylase is selected from the group consisting of the subregions that are defined by amino acid residues 242-246, 250-254, and 268-275 according to the numbering of SEQ ID NO: 133. Preferably, the subregion of region 4 of the amino acid sequence of the amylase is selected from the group consisting of the subregions that are defined by amino acid residues 310-323, 337-345, and 375-378 according to the numbering of SEQ ID NO: 133.

In a preferred embodiment, the amino acid sequence of the amylase of the present invention comprises at least two amino acid substitutions in at least two different regions selected from the group consisting of region 1, region 2, region 3, and region 4. Preferably, the amino acid sequence of the amylase of the present invention comprises at least two amino acid substitutions in at least two different subregions of the regions as described herein selected from the group consisting of region 1, region 2, region 3, and region 4. The at least two substitutions can be in the same or in different subregions of the same region or in different subregions of different regions.

Preferably, the at least one amino acid substitution is a substitution of an amino acid residue present in SEQ ID NO: 133 to an amino acid residue present in SEQ ID NO: 131 and which differs in SEQ ID NO: 131 compared to the amino acid residue present in SEQ ID NO: 133 at this position.

In another embodiment it is preferred to exchange one or more amino acids while at the same time keeping others unchanged to achieve a particular beneficial effect.

In one embodiment, the amino acid sequence of the amylase of the present invention comprises at least one amino acid substitution, preferably, at least two, at least three, or at least four amino acid substitutions, at an amino acid position selected from the group consisting of group consisting of 25, 26, 27, 28, 30, 32, 35, 36, 37, 113, 242, 245, 246, 250, 251, 254, 268, 269, 271, 272, 273, 275, 375, 376, 377, and 378 according to the numbering of SEQ ID NO: 133.

In one embodiment, the amino acid sequence of the amylase of the present invention comprises compared to SEQ ID NO: 133 at least one amino acid substitution, preferably, at least two, at least three, or at least four amino acid substitutions, at an amino acid position selected from the group consisting of group consisting of 25, 26, 27, 28, 30, 32, 35, 36, 37, 113, 242, 245, 246, 250, 251, 254, 268, 269, 271, 272, 273, 275, 375, 376, 377, and 378 according to the numbering of SEQ ID NO: 133.

In one embodiment, the amino acid sequence of the amylase of the present invention comprises at least one amino acid substitution, preferably, at least two, at least three, or at least four amino acid substitutions, selected from the group consisting of T25X, K26X, V27X, K28X, E30X, A32X, S35X, S36X, and L37X, G113X, Y242X, F245X, P246X, T250X, Y251X, N254X, S268X, Y269X, V271X, N272X, K273X, and H275X, K375, Y376, N377, and I378 according to the numbering of SEQ ID NO: 133 (with X being herein any other amino acid).

In one embodiment, the amino acid sequence of the amylase of the present invention comprises at least one amino acid substitution, preferably, at least two, at least three, or at least four amino acid substitutions, selected from the group consisting of T25N; K26R; V27L; K28H,R,A; E30N,D; A32Q,N,T,S,E,G; S35K,A,S; S36N,Q,E,H,D; L37K,A,I, G113Y,Q; Y242F, F245L, P246Q, T250N,G; Y251H; N254G,Q; S268K,Q,N; Y269N; V271L,I; N272G,Q,R; K273A,T; H275E,N; K375S,Q; Y376D,R; N377G,E; and I378V according to the numbering of SEQ ID NO: 133.

In one embodiment, the amino acid sequence of the amylase of the present invention comprises at least one amino acid substitution, preferably, at least two, at least three, or at least four amino acid substitutions, selected from the group consisting of T25N, K26R, V27L, K28H, E30N, A32Q, S35K, S36N, L37A, G113Q Y242F,H; F245L,M; P246Q,K,R; T250N, Y251H, N254G, S268K, Y269N, V271L, N272G, K273A, H275E, K375S, Y376D, N377G, and I378V according to the numbering of SEQ ID NO: 133.

Preferably, the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 1-133 and comprises at least one amino acid substitution, preferably, at least two, at least three, or at least four amino acid substitutions, at an amino acid position selected from the group consisting of group consisting of 25, 26, 27, 28, 30, 32, 35, 36, 37, 113, 242, 245, 246, 250, 251, 254, 268, 269, 271, 272, 273, 275, 375, 376, 377, and 378 according to the numbering of SEQ ID NO: 133.

Preferably, the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 1-102 and comprises at least one amino acid substitution, preferably, at least two, at least three, or at least four amino acid substitutions, at an amino acid position selected from the group consisting of group consisting of 25, 26, 27, 28, 30, 32, 35, 36, 37, 113, 242, 245, 246, 250, 251, 254, 268, 269, 271, 272, 273, 275, 375, 376, 377, and 378 according to the numbering of SEQ ID NO: 133.

Preferably, the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 101 and comprises at least one amino acid substitution, preferably, at least two, at least three, or at least four amino acid substitutions, at an amino acid position selected from the group consisting of group consisting of 25, 26, 27, 28, 30, 32, 35, 36, 37, 113, 242, 245, 246, 250, 251, 254, 268, 269, 271, 272, 273, 275, 375, 376, 377, and 378 according to the numbering of SEQ ID NO: 133.

Preferably, the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 133 and comprises at least one amino acid substitution, preferably, at least two, at least three, or at least four amino acid substitutions, at an amino acid position selected from the group consisting of group consisting of 25, 26, 27, 28, 30, 32, 35, 36, 37, 113, 242, 245, 246, 250, 251, 254, 268, 269, 271, 272, 273, 275, 375, 376, 377, and 378 according to the numbering of SEQ ID NO: 133.

Preferably, the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 133 and comprises compared to SEQ ID NO: 133 at least one amino acid substitution, preferably, at least two, at least three, or at least four amino acid substitutions, at an amino acid position selected from the group consisting of group consisting of 25, 26, 27, 28, 30, 32, 35, 36, 37, 113, 242, 245, 246, 250, 251, 254, 268, 269, 271, 272, 273, 275, 375, 376, 377, and 378 according to the numbering of SEQ ID NO: 133.

Preferably, the amino acid sequence of the amylase of the present invention comprises one or more substitutions as described herein and comprises at least one region, preferably at least two, at least three, most preferably four regions, selected from the regions consisting of region 1, region 2, region 3, and region 4,

wherein region 1 is an amino acid sequence having at least 75%, at least 80%, at least 85%, at least 90%, at least 95%, at least 96%, at least 97%, at least 97.5%, at least 98%, at least 98.5%, at least 99%, or at least 99.5% identity to the amino acid sequence of SEQ ID NO:103, or SEQ ID NO:104; wherein region 2 is an amino acid sequence having at least 75%, at least 80%, at least 85%, at least 90%, at least 95%, at least 96%, at least 97%, at least 97.5%, at least 98%, at least 98.5%, at least 99%, or at least 99.5% identity to an amino acid sequence selected from: SEQ ID NO:105, SEQ ID NO:106, SEQ ID NO:107, SEQ ID NO:108, SEQ ID NO:109, SEQ ID NO:110, SEQ ID NO:111, and SEQ ID NO:112;
wherein region 3 is an amino acid sequence having at least 75%, at least 80%, at least 85%, at least 90%, at least 95%, at least 96%, at least 97%, at least 97.5%, at least 98%, at least 98.5%, at least 99%, or at least 99.5% identity to an amino acid sequence selected from: SEQ ID NO:113, SEQ ID NO:114, SEQ ID NO:115, SEQ ID NO:116, SEQ ID NO:117, SEQ ID NO:118, SEQ ID NO:119, and SEQ ID NO:120; and
wherein region 4 is an amino acid sequence having at least 75%, at least 80%, at least 85%, at least 90%, at least 95%, at least 96%, at least 97%, at least 97.5%, at least 98%, at least 98.5%, at least 99%, or at least 99.5% identity to an amino acid sequence selected from SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, and SEQ ID NO:130.

Preferably, the amylase of the present invention comprises at least one amino acid substitution as described herein and comprises an amino acid sequence that is at least 80%, at least 85%, at least 90%, at least 95%, at least 96%, at least 97%, at least 97.5%, at least 98%, at least 98.5%, at least 99%, or at least 99.5% identical to the amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171.

Preferably, the at least one amino acid substitution is at an amino acid position selected from the group consisting of 113, 242, 245, 246, 268, 269, 271, 272, 273, and 275 according to the numbering of SEQ ID NO: 133.

Preferably, the amino acid sequence of the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 1, 3, 5, 7, 9, 11, 12, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 102, or 133 and comprises at least one amino acid substitution at an amino acid position selected from the group consisting of 113, 242, 245, 246, 268, 269, 271, 272, 273, and 275 according to the numbering of SEQ ID NO: 133.

Preferably, the amino acid sequence of the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 133 and comprises at least one amino acid substitution at an amino acid position selected from the group consisting of 113, 242, 245, 246, 268, 269, 271, 272, 273, and 275 according to the numbering of SEQ ID NO: 133.

Preferably, the at least one amino acid substitution is selected from the group consisting of G113X, Y242X, F245X, P246X, S268X, Y269X, V271X, N272X, K273X, and H275X according to the numbering of SEQ ID NO: 133 (with X being herein any other amino acid).

Preferably, the at least one amino acid substitution is selected from the group consisting of G113Y,Q; Y242F,H; F245L,M; P246Q,K,R; S268K,Q,N; Y269N; V271L,I; N272G,Q,R; K273A,T; and H275E,N according to the numbering of SEQ ID NO: 133.

Preferably, the at least one amino acid substitution is selected from the group consisting of G113Q, Y242F, F245L, P246Q, S268K, Y269N, V271L, N272G, K273A, and H275E according to the numbering of SEQ ID NO: 133.

Preferably, the amino acid sequence of the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 133 and comprises at least one amino acid substitution selected from the group consisting of G113X, Y242X, F245X, P246X, S268X, Y269X, V271X, N272X, K273X, and H275X according to the numbering of SEQ ID NO: 133 (with X being herein any other amino acid).

Preferably, the amino acid sequence of the amylase of the present invention comprises at least two amino acid substitutions at amino acid positions selected from the group consisting of 113; 242, 245, 246, 268, 269, 271, 272, 273, and 275 according to the numbering of SEQ ID NO: 133.

Preferably, the amino acid sequence of the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 1, 3, 5, 7, 9, 11, 12, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 102, or 133 and comprises at least two amino acid substitutions at amino acid positions selected from the group consisting of 113, 242, 245, 246, 268, 269, 271, 272, 273, and 275 according to the numbering of SEQ ID NO: 133.

Preferably, the amino acid sequence of the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 133 and comprises at least two amino acid substitutions at amino acid positions selected from the group consisting of 113, 242, 245, 246, 268, 269, 271, 272, 273, and 275 according to the numbering of SEQ ID NO: 133.

Preferably, the amino acid sequence of the amylase of the present invention comprises at least two amino acid substitutions selected from the group consisting of G113X, Y242X, F245X, P246X, 5268X, Y269X, V271X, N272X, K273X, and H275X according to the numbering of SEQ ID NO: 133 (with X being herein any other amino acid).

Preferably, the at least two amino acid substitutions are selected from the group consisting of G113Y,Q; Y242F,H; F245L,M; P246Q,K,R; S268K,Q,N; Y269N; V271L,I; N272G,Q,R; K273A,T; and H275E,N according to the numbering of SEQ ID NO: 133.

Preferably, the at least two amino acid substitutions are selected from the group consisting of G113Q, Y242F, F245L, P246Q, S268K, Y269N, V271L, N272G, K273A, and H275E according to the numbering of SEQ ID NO: 133.

Preferably, the at least two amino acid substitutions are at least one amino acid substitution selected from the group consisting of G113Y,Q and at least one amino acid substitution selected from the group consisting of Y242F,H; F245L,M; P246Q,K,R; according to the numbering of SEQ ID NO: 133.

Preferably, the at least two amino acid substitutions are at least one amino acid substitution selected from the group consisting of Y242F,H; F245L,M; P246Q,K,R; and at least one amino acid substitution selected from the group consisting of S268K,Q,N; Y269N; V271L,I; N272G,Q,R; K273A,T; and H275E,N according to the numbering of SEQ ID NO: 133.

Preferably, the amino acid sequence of the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 133 and comprises at least two amino acid substitutions selected from the group consisting of G113X, Y242X, F245X, P246X, 5268X, Y269X, V271X, N272X, K273X, and H275X according to the numbering of SEQ ID NO: 133 (with X being herein any other amino acid).

Preferably, the amino acid sequence of the amylase of the present invention comprises two amino acid substitutions at amino acid positions selected from the group consisting of G113, 242, 245, 246, 268, 269, 271, 272, 273, and 275 according to the numbering of SEQ ID NO: 133.

Preferably, the amino acid sequence of the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 1, 3, 5, 7, 9, 11, 12, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 102, or 133 and comprises two amino acid substitutions at amino acid positions selected from the group consisting of 113, 242, 245, 246, 268, 269, 271, 272, 273, and 275 according to the numbering of SEQ ID NO: 133.

Preferably, the amino acid sequence of the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 133 and comprises two amino acid substitutions at amino acid positions selected from the group consisting of 113, 242, 245, 246, 268, 269, 271, 272, 273, and 275 according to the numbering of SEQ ID NO: 133.

Preferably, the amino acid sequence of the amylase of the present invention comprises two amino acid substitution selected from the group consisting of G113X, Y242X, F245X, P246X, S268X, Y269X, V271X, N272X, K273X, and H275X according to the numbering of SEQ ID NO: 133 (with X being herein any other amino acid).

Preferably, the two amino acid substitutions are selected from the group consisting of G113Y,Q; Y242F,H; F245L,M; P246Q,K,R; S268K,Q,N; Y269N; V271L,I; N272G,Q,R; K273A,T; and H275E,N according to the numbering of SEQ ID NO: 133.

Preferably, the two amino acid substitutions are selected from the group consisting of G113Q, Y242F, F245L, P246Q, S268K, Y269N, V271L, N272G, K273A, and H275E according to the numbering of SEQ ID NO: 133.

Preferably, the two amino acid substitutions are one amino acid substitution selected from the group consisting of G113Y,Q and one amino acid substitution selected from the group consisting of Y242F,H; F245L,M; P246Q,K,R; according to the numbering of SEQ ID NO: 133.

Preferably, the two amino acid substitutions are one amino acid substitution selected from the group consisting of Y242F,H; F245L,M; P246Q,K,R; and one amino acid substitution selected from the group consisting of S268K,Q,N; Y269N; V271L,I; N272G,Q,R; K273A,T; and H275E,N according to the numbering of SEQ ID NO: 133.

Preferably, the two amino acid substitutions are at amino acid positions selected from the group consisting of 246/268; 246/273; 268/273, 113/242; 113/245 and 113/246 according to the numbering of SEQ ID NO: 113.

Preferably, the two amino acid substitutions are selected from the group consisting of P246X/S268X; P246X/K273X; S268X/K273X, G113X/Y242X; G113X/F245X and G113X/P246X according to the numbering of SEQ ID NO: 133 (with X being herein any other amino acid).

Preferably, the two amino acid substitutions are selected from the group consisting of P246R,Q,K/S268K,Q,N; P246R,Q,K/K273A,T; S268K,Q,N/K273A,T; G113Y,Q/Y242F,H; G113Y,Q/F245L,M and G113Y,Q/P246Q,K,R according to the numbering of SEQ ID NO: 133.

Preferably, the two amino acid substitutions are selected from the group consisting of P246R/S268K; P246R/K273A; S268K/K273A; G113Q/Y242F; G113Q/F245M and G113Q/P246R according to the numbering of SEQ ID NO: 133.

Preferably, the amino acid sequence of the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 133 and comprises two amino acid substitutions selected from the group consisting of G113X, Y242X, F245X, P246X, S268X, Y269X, V271X, N272X, K273X, and H275X according to the numbering of SEQ ID NO: 133 (with X being herein any other amino acid).

Preferably, the amino acid sequence of the amylase of the present invention comprises three amino acid substitutions at amino acid positions selected from the group consisting of 113, 242, 245, 246, 268, 269, 271, 272, 273, and 275 according to the numbering of SEQ ID NO: 133.

Preferably, the amino acid sequence of the amylase of the present invention has at least 70%, at least 75%, at least 80%, at least 85%, at least 90%, at least 95%, at least 96%, at least 97%, at least 98%, or at least 99% sequence identity to SEQ ID NO: 1, 3, 5, 7, 9, 11, 12, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 102, or 133 and comprises three amino acid substitutions at amino acid positions selected from the group consisting of 113, 242, 245, 246, 268, 269, 271, 272, 273, and 275 according to the numbering of SEQ ID NO: 133.

Preferably, the amino acid sequence of the amylase of the present invention has at least 70%, at least 75%, at least 80%, at least 85%, at least 90%, at least 95%, at least 96%, at least 97%, at least 98%, or at least 99% sequence identity to SEQ ID NO: 133 and comprises three amino acid substitutions at amino acid positions selected from the group consisting of 113, 242, 245, 246, 268, 269, 271, 272, 273, and 275 according to the numbering of SEQ ID NO: 133.

Preferably, the amino acid sequence of the amylase of the present invention comprises three amino acid substitution selected from the group consisting of G113X, Y242X, F245X, P246X, S268X, Y269X, V271X, N272X, K273X, and H275X according to the numbering of SEQ ID NO: 133 (with X being herein any other amino acid).

Preferably, the three amino acid substitutions are selected from the group consisting of G113Y,Q; Y242F,H; F245L,M; P246Q,K,R; S268K,Q,N; Y269N; V271L,I; N272G,Q,R; K273A,T; and H275E,N according to the numbering of SEQ ID NO: 133.

Preferably, the three amino acid substitutions are one amino acid substitution selected from the group consisting of G113Y,Q and two amino acid substitution selected from the group consisting of Y242F,H; F245L,M; P246Q,K,R; according to the numbering of SEQ ID NO: 133.

Preferably, the three amino acid substitutions are one amino acid substitution selected from the group consisting of Y242F,H; F245L,M; P246Q,K,R; and two amino acid substitution selected from the group consisting of S268K,Q,N; Y269N; V271L,I; N272G,Q,R; K273A,T; and H275E,N according to the numbering of SEQ ID NO: 133.

Preferably, the three amino acid substitutions are two amino acid substitution selected from the group consisting of Y242F,H; F245L,M; P246Q,K,R; and one amino acid substitution selected from the group consisting of S268K, Y269N, V271L, N272G, K273A, and H275E, according to the numbering of SEQ ID NO: 133.

Preferably, the three amino acid substitutions are at amino acid positions selected from the group consisting of 246/268/273; 113/242/245; 113/242/246 and 113/245/246 according to the numbering of SEQ ID NO: 133.

Preferably, the three amino acid substitutions are selected from the group consisting of P246X/S268X/K273X; G113X/Y242X/F245X; G113X/F242X/P246X and G113X/F245X/P246X according to the numbering of SEQ ID NO: 133 (with X being herein any other amino acid).

Preferably, the three amino acid substitutions are selected from the group consisting of P246Q,K,R/S268K,Q,N/K273A,T; G113Y,Q/Y242F,H/F245L,M; G113Y,Q/Y242F,H/P246Q,K,R and G113Y,Q/F245L,M/P246Q,K,R according to the numbering of SEQ ID NO: 133.

Preferably, the three amino acid substitutions are selected from the group consisting of P246R/S268K/K273A, G113Q/Y242F/F245M, G113Q/Y242F/P246R and G113Q/F245M/P246R according to the numbering of SEQ ID NO: 133.

Preferably, the amino acid sequence of the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 133 and comprises three amino acid substitutions selected from the group consisting of G113Y,Q; Y242X, F245X, P246X, S268X, Y269X, V271X, N272X, K273X, and H275X according to the numbering of SEQ ID NO: 133 (with X being herein any other amino acid).

Preferably, the amino acid sequence of the amylase of the present invention comprises at least one amino acid substitution at an amino acid position selected from the group consisting of 25, 26, 27, 28, 30, 32, 35, 36, and 37 according to the numbering of SEQ ID NO: 133 while compared to the parent sequence not changing one or more of the amino acid residues at the amino acid position 250, 251, and 254 of SEQ ID NO: 133.

Preferably, the amino acid sequence of the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 1, 3, 5, 7, 9, 11, 12, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 102, or 133 and comprises at least one amino acid substitution at an amino acid position selected from the group consisting of 25, 26, 27, 28, 30, 32, 35, 36, and 37 according to the numbering of SEQ ID NO: 133 while compared to the parent sequence not changing one or more of the amino acid residues at the amino acid position 250, 251, and 254 of SEQ ID NO: 133.

Preferably, the amino acid sequence of the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 133 and comprises at least one amino acid substitution at an amino acid position selected from the group consisting of 25, 26, 27, 28, 30, 32, 35, 36, and 37 according to the numbering of SEQ ID NO: 133 while compared to the parent sequence not changing one or more of the amino acid residues at the amino acid position 250, 251, and 254 of SEQ ID NO: 133.

Preferably, the amino acid sequence of the amylase of the present invention comprises at least one amino acid substitution selected from the group consisting of T25X, K26X, V27X, K28X, E30X, A32X, S35X, S36X, and L37X according to the numbering of SEQ ID NO: 133 while not changing one or more of the amino acid residues T250, Y251, and N254 of SEQ ID NO: 133 (with X being herein any other amino acid).

Preferably, the amino acid sequence of the amylase of the present invention comprises at least one amino acid substitution selected from the group consisting of T25N; K26R; V27L; K28H,R,A; E30N,D; A32Q,N,T,S,E,G; S35K,A,S; S36N,Q,E,H,D; and L37K,A,I according to the numbering of SEQ ID NO: 133 while not changing one or more of the amino acid residues T250, Y251, and N254 of SEQ ID NO: 133.

Preferably, the amino acid sequence of the amylase of the present invention comprises at least one amino acid substitution selected from the group consisting of T25N, K26R, V27L, K28H, E30N, A32Q, S35K, S36N, and L37A according to the numbering of SEQ ID NO: 133 while not changing one or more of the amino acid residues T250, Y251, and N254 of SEQ ID NO: 133.

Preferably, the amino acid sequence of the amylase of the present invention comprises at least one amino acid substitution selected from the group consisting of T25N, K26R, V27L, K28H, E30N, A32Q, S35K, S36N, and L37A according to the numbering of SEQ ID NO: 133 and wherein the amino acid sequence of the amylase comprises one or more of the amino acid residues T250, Y251, and N254 according to the numbering of SEQ ID NO: 133.

Preferably, the amino acid sequence of the amylase of the present invention comprises at least one amino acid substitution at an amino acid position selected from the group consisting of 250, 251, and 254 according to the numbering of SEQ ID NO: 133 while compared to the parent sequence not changing one or more of the amino acid residues at the amino acid position 25, 26, 27, 28, 30, 32, 35, 36, and 37 of SEQ ID NO: 133.

Preferably, the amino acid sequence of the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 1, 3, 5, 7, 9, 11, 12, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 102, or 133 and comprises at least one amino acid substitution at an amino acid position selected from the group consisting of 250, 251, and 254 according to the numbering of SEQ ID NO: 133 while compared to the parent sequence not changing one or more of the amino acid residues at the amino acid position 25, 26, 27, 28, 30, 32, 35, 36, and 37 of SEQ ID NO: 133.

Preferably, the amino acid sequence of the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 133 and comprises at least one amino acid substitution at an amino acid position selected from the group consisting of 250, 251, and 254 according to the numbering of SEQ ID NO: 133 while compared to the parent sequence not changing one or more of the amino acid residues at the amino acid position 25, 26, 27, 28, 30, 32, 35, 36, and 37 of SEQ ID NO: 133.

Preferably, the amino acid sequence of the amylase of the present invention comprises at least one amino acid substitution selected from the group consisting of T250X, Y251X, and N254X according to the numbering of SEQ ID NO: 133 while not changing one or more of the amino acid residues T25, K26, V27, K28, E30, A32, S35, S36, and L37 of SEQ ID NO: 133 (with X being herein any other amino acid).

Preferably, the amino acid sequence of the amylase of the present invention comprises at least one amino acid substitution selected from the group consisting of T250N,G; Y251H; and N254G,Q according to the numbering of SEQ ID NO: 133 while not changing one or more of the amino acid residues T25, K26, V27, K28, E30, A32, S35, S36, and L37 of SEQ ID NO: 133.

Preferably, the amino acid sequence of the amylase of the present invention comprises at least one amino acid substitution selected from the group consisting of T250N, Y251H, and N254G, according to the numbering of SEQ ID NO: 133 while not changing one or more of the amino acid residues T25, K26, V27, K28, E30, A32, S35, S36, and L37 of SEQ ID NO: 133.

Preferably, the amino acid sequence of the amylase of the present invention comprises at least one amino acid substitution selected from the group consisting of T250N, Y251H, and N254G, according to the numbering of SEQ ID NO: 133 and wherein the amino acid sequence of the amylase comprises one or more of the amino acid residues T25, K26, V27, K28, E30, A32, S35, S36, and L37 according to the numbering of SEQ ID NO: 133.

Preferably, the amino acid sequence of the amylase of the present invention comprises at least one amino acid substitution at an amino acid position selected from the group consisting of 25, 26, 27, 28, 30, 32, 35, 36, and 37 according to the numbering of SEQ ID NO: 133 while compared to the parent sequence not changing one or more of the amino acid residues at the amino acid position 375, 376, 377, and 378 of SEQ ID NO: 133.

Preferably, the amino acid sequence of the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 1, 3, 5, 7, 9, 11, 12, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 102, or 133 and comprises at least one amino acid substitution at an amino acid position selected from the group consisting of 25, 26, 27, 28, 30, 32, 35, 36, and 37 according to the numbering of SEQ ID NO: 133 while compared to the parent sequence not changing one or more of the amino acid residues at the amino acid position 375, 376, 377, and 378 of SEQ ID NO: 133.

Preferably, the amino acid sequence of the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 133 and comprises at least one amino acid substitution at an amino acid position selected from the group consisting of 25, 26, 27, 28, 30, 32, 35, 36, and 37 according to the numbering of SEQ ID NO: 133 while compared to the parent sequence not changing one or more of the amino acid residues at the amino acid position 375, 376, 377, and 378 of SEQ ID NO: 133.

Preferably, the amino acid sequence of the amylase of the present invention comprises at least one amino acid substitution selected from the group consisting of T25X, K26X, V27X, K28X, E30X, A32X, S35X, S36X, and L37X according to the numbering of SEQ ID NO: 133 while not changing one or more of the amino acid residues K375, Y376, N377, and I378 of SEQ ID NO: 133 (with X being herein any other amino acid).

Preferably, the amino acid sequence of the amylase of the present invention comprises at least one amino acid substitution selected from the group consisting of T25N; K26R; V27L; K28H,R,A; E30N,D; A32Q,N,T,S,E,G; S35K,A,S; S36N,Q,E,H,D; and L37K,A,I according to the numbering of SEQ ID NO: 133 while not changing one or more of the amino acid residues K375, Y376, N377, and I378 of SEQ ID NO: 133 (with X being herein any other amino acid).

Preferably, the amino acid sequence of the amylase of the present invention comprises at least one amino acid substitution selected from the group consisting of T25N, K26R, V27L, K28H, E30N, A32Q, S35K, S36N, and L37A according to the numbering of SEQ ID NO: 133 while not changing one or more of the amino acid residues K375, Y376, N377, and I378 of SEQ ID NO: 133 (with X being herein any other amino acid).

Preferably, the amino acid sequence of the amylase of the present invention comprises at least one amino acid substitution selected from the group consisting of T25N, K26R, V27L, K28H, E30N, A32Q, S35K, S36N, and L37A according to the numbering of SEQ ID NO: 133 and wherein the amino acid sequence of the amylase comprises one or more of the amino acid residues K375, Y376, N377, and I378 according to the numbering of SEQ ID NO: 133 (with X being herein any other amino acid).

Preferably, the amino acid sequence of the amylase of the present invention comprises at least one amino acid substitution at an amino acid position selected from the group consisting of 375, 376, 377, and 378 according to the numbering of SEQ ID NO: 133 while compared to the parent sequence not changing one or more of the amino acid residues at the amino acid position 25, 26, 27, 28, 30, 32, 35, 36, and 37 of SEQ ID NO: 133.

Preferably, the amino acid sequence of the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 1, 3, 5, 7, 9, 11, 12, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 102, or 133 and comprises at least one amino acid substitution at an amino acid position selected from the group consisting of 375, 376, 377, and 378 according to the numbering of SEQ ID NO: 133 while compared to the parent sequence not changing one or more of the amino acid residues at the amino acid position 25, 26, 27, 28, 30, 32, 35, 36, and 37 of SEQ ID NO: 133.

Preferably, the amino acid sequence of the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 133 and comprises at least one amino acid substitution at an amino acid position selected from the group consisting of 375, 376, 377, and 378 according to the numbering of SEQ ID NO: 133 while compared to the parent sequence not changing one or more of the amino acid residues at the amino acid position 25, 26, 27, 28, 30, 32, 35, 36, and 37 of SEQ ID NO: 133.

Preferably, the amino acid sequence of the amylase of the present invention comprises at least one amino acid substitution selected from the group consisting of K375X, Y376X, N377X, and I378X according to the numbering of SEQ ID NO: 133 while not changing one or more of the amino acid residues T25, K26, V27, K28, E30, A32, S35, S36, and L37 of SEQ ID NO: 133 (with X being herein any other amino acid).

Preferably, the amino acid sequence of the amylase of the present invention comprises at least one amino acid substitution selected from the group consisting of K375S,Q; Y376D,R; N377G,E; and I378V according to the numbering of SEQ ID NO: 133 while not changing one or more of the amino acid residues T25, K26, V27, K28, E30, A32, S35, S36, and L37 of SEQ ID NO: 133 (with X being herein any other amino acid).

Preferably, the amino acid sequence of the amylase of the present invention comprises at least one amino acid substitution selected from the group consisting of K375S, Y376D, N377G, and I378V according to the numbering of SEQ ID NO: 133 while not changing one or more of the amino acid residues T25, K26, V27, K28, E30, A32, S35, S36, and L37 of SEQ ID NO: 133 (with X being herein any other amino acid).

Preferably, the amino acid sequence of the amylase of the present invention comprises at least one amino acid substitution selected from the group consisting of K375S, Y376D, N377G, and I378V according to the numbering of SEQ ID NO: 133 and wherein the amino acid sequence of the amylase comprises one or more of the amino acid residues T25, K26, V27, K28, E30, A32, S35, S36, and L37 according to the numbering of SEQ ID NO: 133 (with X being herein any other amino acid).

Preferably, the amino acid sequence of the amylase of the present invention further comprises a deletion of one or more, preferably two or three, most preferred two amino acid residues, at amino acid positions 180-183, preferably 180-182, according to the numbering of SEQ ID NO: 133. Preferably, the amylase variants of the invention comprise at least one, at least two, or at least three, most preferred two, deletions of amino acid residues selected from the group consisting of R180, S181, T182 and G183, preferably selected from the group consisting of R180, S181, and T182, according to the numbering of SEQ ID NO: 133. These deletions provide improved stability to the amylases.

Preferably, the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 1, 3, 5, 7, 9, 11, 12, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 102, 133, 134, 136, 138, 140, 142, 144, 146, 148, 150, 152, 154, 156, 158, 160, 162, 164, 168, 170, or 171 and comprises at least one amino acid substitution as described herein and at least one, at least two, or at least three, most preferred two, deletions of amino acid residues selected from the group consisting of R180, S181, T182 and G183, preferably selected from the group consisting of R180, S181, and T182, according to the numbering of SEQ ID NO: 133.

Preferably, the amylase of the present invention has at least 70%, at least 75%, at least 80%, at least 85%, at least 90%, at least 95%, at least 96%, at least 97%, at least 97.5%, at least 98%, at least 98.5%, at least 99%, or at least 99.5% sequence identity to SEQ ID NO: 133 and comprises at least one amino acid substitution as described herein and at least one, at least two, or at least three, most preferred two, deletions of amino acid residues selected from the group consisting of R180, S181, T182 and G183, preferably selected from the group consisting of R180, S181, and T182, according to the numbering of SEQ ID NO: 133.

Preferably, the amino acid sequence of the amylase of the present invention comprises a deletion of the C-terminus, preferably as shown in SEQ ID NO: 133. The deletion of the C-terminus of the amino acid sequence of the amylases of the present invention preferably corresponds to a deletion of at least 20, at least 30, at least 40, at least 50, at least 60, at least 70, at least 80, at least 90, or all of amino acid residues 485-583 of the TS23 amylase, which amino acid sequence equals to SEQ ID NO: 133 without the C-terminal deletion.

Preferably, the amino acid sequence of the amylase of the present invention further comprises a deletion of one or more, preferably two amino acid residues, at amino acid positions 180-183 according to the numbering of SEQ ID NO: 133 and a deletion of the C-terminus as shown in SEQ ID NO: 133.

Preferably, the amino acid sequence of the amylase of the present invention may further comprise at least one substitution at a position selected from the group consisting of 87, 98, 109, 130, 149, 178, 185, 189, 194, 201, 225, 242, 243, 272, 282, 309, 320, 359, 439, and 444 according to the numbering of SEQ ID NO: 133.

Preferably, the amino acid sequence of the amylase of the present invention may further comprise at least one of the substitutions selected from the group consisting of Q87X, Q98X, G109X, E130X, G149X, K178X, G185X, E189X, N194X, M201X, N225X, Y242X, S243X, N272X, N282X, R309X, Q320X, Q359X, W439X, and K444X according to the numbering of SEQ ID NO: 133.

Preferably, the amino acid sequence of the amylase of the present invention may further comprise at least one of the substitutions selected from the group consisting of Q87E,R; Q98R; G109A; E130V; G149A; K178L; G185A; E189P; N194F; M201L,I,T; N225E,R; Y242F; S243QA,E,D,N; N272E,R; N282E,R; R309A; Q320R; Q359E; W439R; and K444R according to the numbering of SEQ ID NO: 133.

Preferably, the amylase of the present invention comprises amino acid substitutions at the amino acid positions 242, 245, and 246 according to the numbering of SEQ ID NO: 133 and preferably, the amylase comprising an amino acid sequence that is at least 70% identical to the amino acid sequence of SEQ ID NO: 133.

Preferably, the amylase of the present invention comprises amino acid substitutions Y242F, F245M, and P246R according to the numbering of SEQ ID NO: 133 and preferably, the amylase comprising an amino acid sequence that is at least 70% identical to the amino acid sequence of SEQ ID NO: 133.

Preferably, the amylase of the present invention comprises amino acid substitutions at the amino acid positions marked with “X” according to the numbering of SEQ ID NO: 133 and preferably, wherein the amylase comprises an amino acid sequence that is at least 70% identical to the amino acid sequence of SEQ ID NO: 133 (with X being herein any other amino acid and empty box being no substitution):

| 25 | 26 | 27 | 28 | 30 | 32 | 35 | 36 | 37 | 113 | 242 | 245 | 246 | 268 | 269 | 271 | 272 | 273 | 275 | 375 | 376 | 377 | 378 |
|---|---|---|---|---|---|---|---|---|---|---|---|---|---|---|---|---|---|---|---|---|---|---|
| X | X | X | X | X | X | X | X | X | | X | X | X | X | X | X | X | X | X | | | | |
| | | | | | | | | | X | X | X | X | | | | | | | | | | |
| X | X | X | X | X | X | X | X | X | X | X | X | X | | | | | | | X | X | X | X |
| X | X | X | X | X | X | X | X | X | | X | X | X | X | X | X | X | X | X | | | | |
| X | X | X | X | X | X | X | X | X | | X | X | X | X | X | X | X | X | X | | | | |
| X | X | X | X | X | X | X | X | X | | X | X | X | X | X | X | X | X | X | | | | |
| X | X | X | X | X | X | X | X | X | X | X | X | X | | | | | | | X | X | X | X |
| X | X | X | X | X | X | X | X | X | | X | X | X | X | X | X | X | X | X | | | | |
| | | | | | | | | | | | | X | X | | | | X | | | | | |

Preferably, the amylase of the present invention comprises the following amino acid residues at the following amino acid positions according to the numbering of SEQ ID NO: 133 and preferably, wherein the amylase comprises an amino acid sequence that is at least 70% identical to the amino acid sequence of SEQ ID NO: 133 (with empty box being no substitution):

| 25 | 26 | 27 | 28 | 30 | 32 | 35 | 36 | 37 | 113 | 242 | 245 | 246 | 268 | 269 | 271 | 272 | 273 | 275 | 375 | 376 | 377 | 378 |
|---|---|---|---|---|---|---|---|---|---|---|---|---|---|---|---|---|---|---|---|---|---|---|
| N | R | L | H | N | Q | K | N | A | | F | M | R | K | N | L | G | A | E | | | | |
| | | | | | | | | | Q | F | M | R | | | | | | | | | | |
| N | R | L | H | N | Q | K | N | A | Q | F | M | R | | | | | | | S | D | G | V |
| N | R | L | H | N | Q | K | N | A | | F | M | R | K | N | L | G | A | E | | | | |
| N | R | L | H | N | Q | K | N | A | | F | M | R | K | N | L | G | A | E | | | | |
| N | R | L | H | N | Q | K | N | A | | F | M | R | K | N | L | G | A | E | | | | |
| N | R | L | H | N | Q | K | N | A | Q | F | M | R | | | | | | | S | D | G | V |
| N | R | L | H | N | Q | K | N | A | | F | M | R | K | N | L | G | A | E | | | | |
| | | | | | | | | | | | | R | K | | | | A | | | | | |

### DETAILED DESCRIPTION OF THE INVENTION

An enzyme is a biological molecule (polypeptide) comprising a sequence of amino acid residues, wherein the enzyme can catalyze a reaction. Hence, enzymes are catalytically active proteins or polypeptides. Enzyme names are determined based on the recommendations of the Nomenclature Committee of the International Union of Biochemistry and Molecular Biology (IUBMB). Enzymes are defined by an EC (Enzyme Commission) number, recommended name, alternative names (if any), catalytic activity, and other factors. Enzymes herein may be identified by polypeptide sequences (also called amino acid sequences herein). The polypeptide sequence specifies the three-dimensional structure including the “active site” of an enzyme which in turn determines the catalytic activity of the same. Polypeptide sequences may be identified by a SEQ ID NO.

Enzymes are obtained from or derived from many different sources including: plants; animals; bacteria, archaea, fungi, yeast, environmental samples containing DNA that encodes an enzyme, or enzymes can be synthetic generated in a laboratory. For example, bacterial sources of enzymes include enzymes derived from Bacillus, Streptomyces, E. coli and Pseudomonas; fungal sources of enzymes include enzymes derived from Aspergillus, Fusarium, Thermomyces and Trichoderma; yeast sources of enzymes include enzymes derived from Pichia, and Saccharomyces.

Different classes of enzymes are known to be useful in detergents and cleaning products including: lipase, amylase, protease, cellulase, mannanase, pectate lyase, and nuclease; however, there is a need in the industry to provide an amylase that has more activity, temperature profile, pH profile, has improved performance (stain removal), stability in presence of protease, or a combination thereof. The amylase enzymes of the invention address the industrial needs.

The World Intellectual Property Office (WIPO) Standard ST.25 (1998) provides that the amino acid residues should be represented in the sequence listing using the following three-letter symbols with the first letter as a capital. The table below provides an overview of the amino acid identifiers as well as the corresponding DNA codons that encode the amino acid using the standard genetic standard. The DNA codons that encode amino acid residues can be different depending organism that is used and slightly different tables for translation of the genetic code may apply. A compilation of such non-standard code translation tables is maintained at the NCBI.

A “parent” polypeptide amino acid sequence is the starting sequence for introduction of mutations (e.g. by introducing one or more (fragments) amino acid substitutions, insertions, deletions, or a combination thereof) to the sequence, resulting in “variants” of the parent polypeptide amino acid sequences. A parent includes: A wild-type polypeptide amino acid sequence or synthetically generated polypeptide amino acid sequence that is used as starting sequence for introduction of (further) changes.

A “variant polypeptide” refers to an enzyme that differs from its parent in its amino acid sequence. The differences between the parent polypeptide and variant polypeptide can be one single amino acid residue, or more than one amino acid residue. The more than one amino acid residue and be consecutive amino acid residues or non-consecutive amino acid residues. The consecutive amino acid residues can be four consecutive amino acid residues; five consecutive amino acid residues; eight consecutive amino acid residues; nine consecutive amino acid residues; eleven consecutive amino acid residues; thirteen consecutive amino acid residues; or fourteen consecutive amino acid residues. While the definition below describes variants in the context of amino acid changes, nucleic acids may be similarly modified, e.g. by substitutions.

A “mature polypeptide” means an enzyme in its final form including any post-translational modifications, glycosylation, phosphorylation, truncation, N-terminal modifications, C-terminal modifications, signal sequence deletion. A mature polypeptide can vary depending upon the expression system, vector, promoter, and/or production process.

A “synthetic” or “artificial” compound is produced by in vitro chemical or enzymatic synthesis.

The term “non-naturally occurring” refers to a (poly)nucleotide, amino acid, (poly)peptide, enzyme, protein, cell, organism, or other material that is not present in its original naturally occurring environment or source. Preferably, the amylase of the present invention is a non-naturally occurring amylase.

Variant polynucleotide and variant polypeptide sequences may be defined by their sequence identity when compared to a parent sequence. Sequence identity usually is provided as “% sequence identity” or “% identity”. For calculation of sequence identities, in a first step a sequence alignment is produced. According to this invention, a pairwise global alignment is produced, meaning that two sequences are aligned over their complete length, which is usually produced by using a mathematical approach, called alignment algorithm.

According to the invention, the alignment is generated by using the algorithm of Needleman and Wunsch (J. Mol. Biol. (1979) 48, p. 443-453). Preferably, the program “NEEDLE” (The European Molecular Biology Open Software Suite (EMBOSS)) is used for the purposes of the current invention, with using the programs default parameter (polynucleotides: gap open=10.0, gap extend=0.5 and matrix=EDNAFULL; polypeptides: gap open=10.0, gap extend=0.5 and matrix=EBLOSUM62).

After aligning two sequences, in a second step, an identity value is determined from the alignment produced.

For this purpose, the %-identity is calculated by dividing the number of identical residues by the length of the alignment region which is showing the respective sequence of the present invention over its complete length multiplied with 100: %−identity=(identical residues/length of the alignment region which is showing the respective sequence of the present invention over its complete length)*100.

For calculating the percent identity of two nucleic acid sequences the same applies as for the calculation of percent identity of two amino acid sequences with some specifications. For nucleic acid sequences encoding for a protein the pairwise alignment shall be made over the complete length of the coding region of the sequence of this invention from start to stop codon excluding introns. Introns present in the other sequence, to which the sequence of this invention is compared, may also be removed for the pairwise alignment. Percent identity is then calculated by %−identity=(identical residues/length of the alignment region which is showing the sequence of the invention from start to stop codon excluding introns over their complete length)*100. After aligning two sequences, in a second step, an identity value is determined from the alignment produced.

Moreover, the preferred alignment program for nucleic acid sequences implementing the Needleman and Wunsch algorithm (J. Mol. Biol. (1979) 48, p. 443-453) is “NEEDLE” (The European Molecular Biology Open Software Suite (EMBOSS)) with the programs default parameters (gapopen=10.0, gapextend=0.5 and matrix=EDNAFULL).

Sequences, having identical or similar regions with a sequence of this invention, and which shall be compared with a sequence of this invention to determine % identity, can easily be identified by various ways that are within the skill in the art, for instance, using publicly available computer methods and programs such as BLAST, BLAST-2, available for example at NCBI.

Variant polypeptides may be defined by their sequence similarity when compared to a parent sequence. Sequence similarity usually is provided as “% sequence similarity” or “%-similarity”. % sequence similarity takes into account that defined sets of amino acids share similar properties, e.g. by their size, by their hydrophobicity, by their charge, or by other characteristics. Herein, the exchange of one amino acid with a similar amino acid may be called “conservative mutation”. Similar amino acids according to the invention are defined as follows, which shall also apply for determination of %-similarity according to this invention, which is also in accordance with the BLOSUM62 matrix as for example used by program “NEEDLE”, which is one of the most used amino acids similarity matrix for database searching and sequence alignments:

Amino acid A is similar to amino acids S
Amino acid D is similar to amino acids E; N
Amino acid E is similar to amino acids D; K; Q
Amino acid F is similar to amino acids W; Y
Amino acid H is similar to amino acids N; Y
Amino acid I is similar to amino acids L; M; V
Amino acid K is similar to amino acids E; Q; R
Amino acid L is similar to amino acids I; M; V
Amino acid M is similar to amino acids I; L; V
Amino acid N is similar to amino acids D; H; S
Amino acid Q is similar to amino acids E; K; R
Amino acid R is similar to amino acids K; Q
Amino acid S is similar to amino acids A; N; T
Amino acid T is similar to amino acids S
Amino acid V is similar to amino acids I; L; M
Amino acid W is similar to amino acids F; Y
Amino acid Y is similar to amino acids F; H; W

Conservative amino acid substitutions may occur over the full length of the sequence of a polypeptide sequence of a functional protein such as an enzyme. In one embodiment, such mutations are not pertaining the functional domains of an enzyme. In one embodiment, conservative mutations are not pertaining the catalytic centers of an enzyme.

For calculation of sequence similarity, in a first step a sequence alignment is produced as described above. After aligning two sequences, in a second step, a similarity value is determined from the alignment produced.

For this purpose, the %-similarity is calculated by dividing the number of identical residues plus the number of similar residues by the length of the alignment region which is showing the sequence of the invention over its complete length multiplied with 100: %−similarity=[(identical residues+similar residues)/length of the alignment region which is showing the sequence of the invention over its complete length]*100.

The invention relates to a polypeptide having amylase activity comprising an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to any one of the full length amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171.

The invention relates to a polypeptide having amylase activity comprising an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to any one of the full length amino acid sequence of SEQ ID NO: 134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171.

The invention relates to a polypeptide having amylase activity comprising an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to any one of the full length amino acid sequence of SEQ ID NO:1, SEQ ID NO:11, SEQ ID NO:15, SEQ ID NO:146, SEQ ID NO:148, SEQ ID NO: 152, SEQ ID NO: 154; SEQ ID NO: 170, or SEQ ID NO:171.

The invention relates to a polypeptide having amylase activity comprising an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to any one of the full length amino acid sequence of SEQ ID NO:11. The invention relates to a polypeptide having amylase activity comprising an amino acid sequence that is at least 75% identical, 76% identical, at least 77% identical, at least 78% identical, at least 79% identical, at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to any of the full length amino acid sequence of SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, or SEQ ID NO:130.

The invention further relates to a polynucleotide encoding a variant polypeptide of the invention. The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length. A “gene” is a DNA segment carrying a certain genetic information.

A “parent” polynucleotide acid sequence is the starting sequence for introduction of mutations to the sequence, resulting in “variants” of said parent polynucleotide sequence. A “variant polynucleotide” refers to a polynucleotide that encodes an enzyme and the variant polynucleotide differs from its parent polynucleotide in its nucleic acid sequence.

The polynucleotide of the invention in one aspect has a nucleic acid sequence which is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical when compared to any one of the full length polynucleotide sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:68, SEQ ID NO:70, SEQ ID NO:72, SEQ ID NO:74, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, SEQ ID NO:161, SEQ ID NO:163, SEQ ID NO:165, SEQ ID NO:167, or SEQ ID NO:169.

The polynucleotide of the invention in one aspect has a nucleic acid sequence which is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical when compared to any one of the full length polynucleotide sequence of SEQ ID NO:2, SEQ ID NO: 12, SEQ ID NO:16, SEQ ID NO:147, SEQ ID NO:149, SEQ ID NO: 155, or SEQ ID NO: 153.

In one embodiment, the invention relates to a polypeptide having amylase activity which is encoded by a polynucleotide having a nucleic acid sequence at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, at least 99.5%, or 100% identical to any one of the full length polynucleotide sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:68, SEQ ID NO:70, SEQ ID NO:72, SEQ ID NO:74, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, SEQ ID NO:161, SEQ ID NO:163, SEQ ID NO:165, SEQ ID NO:167, or SEQ ID NO:169.

Preferably, the polynucleotide is a codon-optimized polynucleotide for improving expression in a specific host cell.

“Substitutions” are described by providing the original amino acid followed by the number of the position within the amino acid sequence, followed by the substituted amino acid. A specific amino acid residue may be substituted with any of the 19 amino acid residues different from the original one. For example, the substitution of histidine at position 120 with alanine is designated as “His120Ala” or “H120A”. Alternative substitutions at an amino acid position are indicated as follows “His120Ala,Leu” or “H120A,L”. It is understood herein that instead of the indicated specific substitutions alternative substitutions using conservative amino acid alternatives can be used.

Amino acid deletions are described by providing the original amino acid of the parent enzyme followed by the number of the position within the amino acid sequence, followed by *. Accordingly, the deletion of glycine at position 150 is designated as “Gly150*” or G150*”. Alternatively, deletions are indicated by e.g. “deletion of D183 and G184”.

Preferably, the amino acid sequence of the amylase of the present invention comprises a deletion of one or more, preferably two or three, most preferred two amino acid residues, at amino acid positions 180-183 according to the numbering of SEQ ID NO: 133. Preferably, the amylase variants of the invention comprise at least one, at least two, or at least three, most preferred two, deletions of amino acid residues selected from the group consisting of R180, S181, T182 and G183 according to the numbering of SEQ ID NO: 133. These deletions provide improved stability to the amylases.

Amino acid insertions are described by providing the original amino acid of the parent enzyme followed by the number of the position within the amino acid sequence, followed by the original amino acid and the additional amino acid. For example, an insertion at position 180 of lysine next to glycine is designated as “Gly180GlyLys” or “G180GK”. When more than one amino acid residue is inserted, such as e.g. a Lys and Ala after Gly180 this may be indicated as: Gly180GlyLysAla or G195GKA.

In cases where a substitution and an insertion occur at the same position, this may be indicated as S99SD+S99A or in short S99AD.

The one or more amino acid substitution of the variant polypeptides can be one or more conservative amino acid substitution. A “conservative amino acid substitution” or “related amino acid” means replacement of one amino acid residue in an amino acid sequence with a different amino acid residue having a similar property at the same position compared to the parent amino acid sequence. Some examples of a conservative amino acid substitution include but are not limited to replacing a positively charged amino acid residue with a different positively charged amino acid residue; replacing a polar amino acid residue with a different polar amino acid residue; replacing a non-polar amino acid residue with a different non-polar amino acid residue, replacing a basic amino acid residue with a different basic amino acid residue, or replacing an aromatic amino acid residue with a different aromatic amino acid residue.

“Enzymatic activity” means at least one catalytic effect exerted by an enzyme. Enzymatic activity is expressed as units per milligram of enzyme (specific activity) or molecules of substrate transformed per minute per molecule of enzyme (molecular activity). Enzymatic activity can be specified by the enzymes actual function, e.g. proteases exerting proteolytic activity by catalyzing hydrolytic cleavage of peptide bonds, lipases exerting lipolytic activity by hydrolytic cleavage of ester bonds, amylases activity involves (endo)hydrolysis of glucosidic linkages in polysaccharides, etc.

Enzymatic activity may change during storage or operational use of the enzyme. The term “enzyme stability” relates to the retention of enzymatic activity as a function of time during storage or operation. The term “storage” herein means to indicate the fact of products or compositions or formulations being stored from the time of being manufactured to the point in time of being used in final application. Retention of enzymatic activity as a function of time during storage may be called “storage stability” herein.

To determine and quantify changes in catalytic activity of enzymes stored or used under certain conditions over time, the “initial enzymatic activity” is measured under defined conditions at time zero (100%) and at a certain point in time later (x %). By comparison of the values measured, a potential loss of enzymatic activity can be determined in its extent. The extent of enzymatic activity loss determines an enzymes stability or non-stability.

Parameters influencing the enzymatic activity of an enzyme and/or storage stability and/or operational stability are for example pH, temperature, chelators, and presence of oxidative substances.

A variant polypeptide may be active over a broad pH at any single point within the range from about pH 4.0 to about pH 12.0. The variant polypeptides enzyme may be active over a range of pH 5.0 to pH 11.0, pH 6.0 to pH 10.0, and pH 7.0 to pH 9.0. In another embodiment, the variant polypeptides enzyme may be active over a pH 7.1 to pH 8.9, pH 7.2 to pH 8.8, pH 7.3 to pH 8.7, pH 7.4 to pH 8.6, pH 7.5 to pH 8.5. The variant polypeptides may be active at pH 4.0, pH 4.1, pH 4.2, pH 4.3, pH 4.4, pH 4.5, pH 4.6, pH 4.7, pH 4.8, pH 4.9, pH 5.0, pH 5.1, pH 5.2, pH 5.3, pH 5.4, pH 5.5, pH 5.6, pH 5.7, pH 5.8, pH 5.9, pH 6.0, pH 6.1, pH 6.2, pH 6.3, pH 6.4, pH 6.5, pH 6.6, pH 6.7, pH 6.8, pH 6.9, pH 7.0, pH 7.1, pH 7.2, pH 7.3, pH 7.4, pH 7.5, pH 7.6, pH 7.7, pH 7.8, pH 7.9, pH 8.0, pH 8.1, pH 8.2, pH 8.3, pH 8.4, pH 8.5, pH 8.6 pH 8.7, pH 8.8 pH 8.9, pH 9.0, pH 9.1, pH 9.2, pH 9.3, pH 9.4, pH 9.5, pH 9.6, pH 9.7, pH 9.8, pH 9.9, pH 10.0, pH 10.1, pH 10.2, pH 10.3, pH 10.4, pH 10.5, pH 10.6, pH 10.7, pH 10.8, pH 10.9, pH 11.0, pH 11.1, pH 11.2, pH 11.3, pH 11.4, pH 11.5, pH 11.6, pH 11.7, pH 11.8, pH 11.9, pH 12.0, pH 12.1, pH 12.2, pH 12.3, pH 12.4, and pH 12.5, pH 12.6, pH 12.7, pH 12.8, pH 12.9, or higher.

A “pH stability”, refers to the ability of an enzyme to exert enzymatic activity at a specific pH range.

The variant polypeptides may be active over a broad temperature, wherein the temperature is any point in the range from about 10° C. to about 95° C. The variant polypeptides may be active at a temperature range from 10° C. to 55° C., 10° C. to 50° C., 10° C. to 45° C., 10° C. to 40° C., 10° C. to 35° C., 10° C. to 30° C., or 10° C. to 25° C. The variant polypeptides may be active at a temperature range from 20° C. to 55° C., 20° C. to 50° C., 20° C. to 45° C., 20° C. to 40° C., 20° C. to 35° C., 20° C. to 30° C., or 20° C. to 25° C. The variant polypeptides are active at a temperature of at least 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., or higher temperatures.

The terms “thermal stability” and “thermostability” refer to the ability of a protein to exert catalytic activity at a specific temperature range. Enzymes thermostability may be characterized by what is known as the T50 value (also called half-life, see above). The T50 indicates the temperature at which 50% residual enzymatic activity is still present after thermal inactivation for a certain time when compared with a reference sample which has not undergone thermal treatment.

In one embodiment, the variant polypeptides improve the thermostability compared to the parent molecule. In another embodiment the variant polypeptides improve the thermostability by 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., or more degrees C. when compared to the parent polypeptide. In another embodiment, the thermostability increase is measured at a temperature between 70° C. and 100° C. The thermostability increase can be measured at 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., and/or 100° C. In another embodiment, the thermostability increase is measured at a temperature of 80° C. In another embodiment, the thermostability increase is measured a temperature of 90° C.

In one embodiment, the variant polypeptide is a fragment of the full-length amino acid sequence and the fragment has amylase activity.

A “Fragment”, or “subsequence” as used herein are a portion of a polynucleotide or an amino acid sequence.

The term “functional fragment” refers to any nucleic acid or amino acid sequence which comprises merely a part of the full-length amino acid sequence, respectively, but still has the same or similar activity and/or function. Preferably, the functional fragment is at least 75% identical, at least 76% identical, at least 77% identical, at least 78% identical, at least 79% identical, at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, or at least 99.5% identical to the full length amino acid sequence original sequence. The functional fragment comprises contiguous nucleic acids or amino acids compared to the original nucleic acid or original amino acid sequence, respectively.

The polypeptide variants can be a hybrid of more than one amylase enzymes. In one embodiment, the polypeptide variant can be a hybrid of the polypeptide variant and at least one additional enzyme selected from the group consisting of: a second amylase, a lipase, a protease, a cellulase, a laccase, a pectinase, and a nuclease, wherein the hybrid has amylase activity and enzymatic activity of the second enzyme.

In one embodiment, the present invention refers to a polypeptide comprising a hybrid of at least one polypeptide as described herein and a second polypeptide having an activity selected from the group consisting of amylase activity, wherein the hybrid also has amylase activity.

In on embodiment the variant polypeptide comprises a hybrid of at least one variant polypeptide as described herein, and a second polypeptide having amylase activity, wherein the hybrid has amylase activity.

A “hybrid” or “chimeric” or “fusion protein” means that a fragment of the amino acid sequence of a first enzyme is combined with a fragment of the amino acid sequence of a second enzyme to form a hybrid enzyme wherein the hybrid has an enzyme activity.

The hybrid enzymes can be engineered with fragments from amino acid sequences of more than two enzymes. The domain of an amylase of this invention may be combined with a domain of a commercially available amylase such as: Duramyl™, Termamyl™, Termamyl SC™, Termamyl Ultra™, Fungamyl™, Stainzyme™, Stainzyme Plus™, Natalase™, Liquozyme X, Supramyl™ Amplify™, Amplify Prime™ and BAN™ (from Novozymes A/S), and Rapidase™, Purastar™ Purastar OxAm™ Powerase™, Effectenz™ (M100 from DuPont), Preferenz™ (S1000, S110 and F1000; from DuPont), PrimaGreen™ (ALL; DuPont), Optisize™ (DuPont) and Kam™ (Kao) and Kemzyme™ (Biozym), to form a hybrid enzyme and the hybrid has amylase activity.

In another aspect the present invention refers to a composition comprising the polypeptide described herein. A composition may comprise combinations of the polypeptides with another enzyme. The combination of enzymes can be of the same class, for example a composition comprising a first amylase and a second amylase. Combinations of enzymes can be from a different class of enzymes, for example, a composition comprising a lipase and an amylase. Combinations of enzymes can be compositions comprising at least one amylase of the invention and one or more second enzymes. In one embodiment, the composition comprises one second enzyme, two second enzymes, three second enzymes, four second enzymes, or more than four second enzymes. In an embodiment, the second enzyme is selected from the group consisting of: a second amylase, a lipase, a protease, a cellulase, a laccase, a pectinase, and a nuclease, or any combination thereof.

Additional enzymes suitable for the hybrid or the composition of the present invention are further described below. In one embodiment, suitable enzymes include enzyme variants having enzymatic activity which are at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% identical when compared to the full length polypeptide sequence of the parent enzyme as disclosed below.

### Amylase

Alpha-amylase (E.C. 3.2.1.1) enzymes may perform endohydrolysis of (1->4)-alpha-D-glucosidic linkages in polysaccharides containing three or more (1->4)-alpha-linked D-glucose units. Amylase enzymes act on starch, glycogen and related polysaccharides and oligosaccharides in a random manner; reducing groups are liberated in the alpha-configuration. Other examples of amylase enzymes include: Beta-amylase (E.C. 3.2.1.2), Glucan 1,4-alpha-maltotetraohydrolase (E.C. 3.2.1.60), Isoamylase (E.C. 3.2.1.68), Glucan 1,4-alpha-maltohexaosidase (E.C. 3.2.1.98), and Glucan 1,4-alpha-maltohydrolase (E.C. 3.2.1.133).

The amylases described below can be used as a parent amylase to create variant amylases by introducing one or more amino acid substitutions and/or deletions as described herein.

Many amylase enzymes have been described in patents and published patent applications including, but not limited to: WO 2002/068589, WO 2002/068597, WO 2003/083054, WO 2004/091544, and WO 2008/080093.

Amylases are known to be derived from Bacillus licheniformis having SEQ ID NO:2 as described in WO 95/10603. Suitable variants are those which are at least 90% identical to SEQ ID NO: 2 as described in WO 95/10603 and/or comprising one or more substitutions in the following positions: 15, 23, 105, 106, 124, 128, 133, 154, 156, 178, 179, 181, 188, 190, 197, 201, 202, 207, 208, 209, 211, 243, 264, 304, 305, 391, 408, and 444 which have amylolytic activity. Such variants are described in WO 94/02597, WO 94/018314, WO 97/043424 and SEQ ID NO:4 of WO 99/019467.

Amylases are known to be derived from B. stearothermophilus having SEQ ID NO:6 as described in WO 02/10355 or an amylase which is at least 90% identical thereto having amylolytic activity with optionally having a C-terminal truncation over the wildtype sequence. Suitable variants of SEQ ID NO:6 include those which is at least 90% identical thereto and/or further comprise a deletion in positions 181 and/or 182 and/or a substitution in position 193.

Amylases are known to be derived from Bacillus sp. 707 having SEQ ID NO:6 as disclosed in WO 99/19467 or an amylase which is at least 90% identical thereto having amylolytic activity. Amylases are known from Bacillus halmapalus having SEQ ID NO:2 or SEQ ID NO:7 as described in WO 96/23872, also described as SP-722, or an amylase which is at least 90% identical to one of the sequences which has amylolytic activity.

Amylases are known to be derived from Bacillus sp. DSM 12649 having SEQ ID NO:4 as disclosed in WO 00/22103 or an amylase which is at least 90% identical thereto having amylolytic activity.

Amylases are known from Bacillus strain TS-23 having SEQ ID NO:2 as disclosed in WO 2009/061380 or an amylase which is at least 90% identical thereto having amylolytic activity. Amylases are known from Cytophaga sp. having SEQ ID NO:1 as disclosed in WO 2013/184577 or an amylase which is at least 90% identical thereto having amylolytic activity.

Amylases are known from Bacillus megaterium DSM 90 having SEQ ID NO:1 as disclosed in WO 2010/104675 or an amylase which is at least 90% identical thereto having amylolytic activity.

Amylases are known having amino acids 1 to 485 of SEQ ID NO:2 as described in WO 00/60060 or amylases comprising an amino acid sequence which is at least 96% identical with amino acids 1 to 485 of SEQ ID NO:2 which have amylolytic activity.

Amylases are also known having SEQ ID NO: 12 as described in WO 2006/002643 or amylases having at least 80% identity thereto and have amylolytic activity. Suitable amylases include those having at least 80% identity compared to SEQ ID NO:12 and/or comprising the substitutions at positions Y295F and M202LITV and have amylolytic activity.

Amylases are also known having SEQ ID NO:6 as described in WO 2011/098531 or amylases having at least 80% identity thereto having amylolytic activity. Suitable amylases include those having at least 80% identity compared to SEQ ID NO:6 and/or comprising a substitution at one or more positions selected from the group consisting of 193 [G,A,S,T or M], 195 [F,W,Y,L,I or V], 197 [F,W,Y,L,I or V], 198 [Q or N], 200 [F,W,Y,L,I or V], 203 [F,W,Y,L,I or V], 206 [F,W,Y,N,L,I,V,H,Q,D or E], 210 [F,W,Y,L,I or V], 212 [F,W,Y,L,I or V], 213 [G,A,S,T or M] and 243 [F,W,Y,L,I or V] and have amylolytic activity.

Amylases are known having SEQ ID NO:1 as described in WO 2013/001078 or amylases having at least 85% identity thereto having amylolytic activity. Suitable amylases include those having at least 85% identity compared to SEQ ID NO:1 and/or comprising an alteration at two or more (several) positions corresponding to positions G304, W140, W189, D134, E260, F262, W284, W347, W439, W469, G476, and G477 and having amylolytic activity.

Amylases are known having SEQ ID NO:2 as described in WO 2013/001087 or amylases having at least 85% identity thereto and having amylolytic activity. Suitable amylases include those having at least 85% identity compared to SEQ ID NO:2 and/or comprising a deletion of positions 181+182, or 182+183, or 183+184 according to the numbering of SEQ ID NO: 2 as described in WO 2013/001087, which have amylolytic activity. Suitable amylases include those having at least 85% identity compared to SEQ ID NO:2 and/or comprising a deletion of positions 181+182, or 182+183, or 183+184 according to the numbering of SEQ ID NO: 2 as described in WO 2013/001087, which comprise one or two or more modifications in any of positions corresponding to W140, W159, W167, Q169, W189, E194, N260, F262, W284, F289, G304, G305, 8320, W347, W439, W469, G476 and G477 and have amylolytic activity.

Amylases also include hybrid α-amylase from above mentioned amylases as for example as described in WO 2006/066594.

Commercially available amylase enzymes include: Duramyl™, Termamyl™, Termamyl SC™, Termamyl Ultra™, Fungamyl™, Stainzyme™, Stainzyme Plus™, Natalase™, Liquozyme X, Supramyl™ Amplify™, Amplify Prime™ and BAN™ (from Novozymes A/S), and Rapidase™ Purastar™, Purastar OxAm™ Powerase™, Effectenz™ (M100 from DuPont), Preferenz™ (S1000, S110 and F1000; from DuPont), PrimaGreen™ (ALL; DuPont), Optisize™ (DuPont) and Kam™ (Kao) and Kemzyme™ (Biozym).

### Lipase

“Lipases”, “lipolytic enzyme”, “lipid esterase”, all refer to an enzyme of EC class 3.1.1 (“carboxylic ester hydrolase”). Lipases (E.C. 3.1.1.3, Triacylglycerol lipase) may hydrolyze triglycerides to more hydrophilic mono- and diglycerides, free fatty acids, and glycerol. Lipase enzymes usually includes also enzymes which are active on substrates different from triglycerides or cleave specific fatty acids, such as Phospholipase A (E.C. 3.1.1.4), Galactolipase (E.C. 3.1.1.26), cutinase (EC 3.1.1.74), and enzymes having sterol esterase activity (EC 3.1.1.13) and/or wax-ester hydrolase activity (EC 3.1.1.50).

Many lipase enzymes have been described in patents and published patent applications including, but not limited to: WO2000032758, WO2003/089620, WO2005/032496, WO2005/086900, WO200600976, WO2006/031699, WO2008/036863, WO2011/046812, and WO2014059360.

Lipases are used in detergent and cleaning products to remove grease, fat, oil, and dairy stains. Commercially available lipases include but are not limited to: Lipolase™, Lipex™, Lipolex™ and Lipoclean™ (Novozymes A/S), Lumafast (originally from Genencor) and Lipomax (Gist-Brocades/now DSM).

The methods for determining lipolytic activity are well-known in the literature (see e.g. Gupta et al. (2003), Biotechnol. Appl. Biochem. 37, p. 63-71). E.g. the lipase activity may be measured by ester bond hydrolysis in the substrate para-nitrophenyl palmitate (pNP-Palmitate, C:16) and releases pNP which is yellow and can be detected at 405 nm.

### Protease

Enzymes having proteolytic activity are called “proteases” or “peptidases”. Proteases are active proteins exerting “protease activity” or “proteolytic activity”.

Proteases are members of class EC 3.4. Proteases include aminopeptidases (EC 3.4.11), dipeptidases (EC 3.4.13), dipeptidyl-peptidases and tripeptidyl-peptidases (EC 3.4.14), peptidyl-dipeptidases (EC 3.4.15), serine-type carboxypeptidases (EC 3.4.16), metallocarboxypeptidases (EC 3.4.17), cysteine-type carboxypeptidases (EC 3.4.18), omega peptidases (EC 3.4.19), serine endopeptidases (EC 3.4.21), cysteine endopeptidases (EC 3.4.22), aspartic endopeptidases (EC 3.4.23), metallo-endopeptidases (EC 3.4.24), threonine endopeptidases (EC 3.4.25), endopeptidases of unknown catalytic mechanism (EC 3.4.99).

Commercially available protease enzymes include but are not limited to Lavergy™ Pro (BASF); Alcalase®, Blaze®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® (Novozymes A/S), those sold under the tradename Maxatase®, Maxacal®, Maxapem®, Purafect®, Purafect® Prime, Purafect MA®, Purafect Ox®, Purafect OxP®, Puramax®, Properase®, FN2®, FN3®, FN4®, Excellase®, Eraser®, Ultimase®, Opticlean®, Effectenz®, Preferenz® and Optimase® (Danisco/DuPont), Axapem™ (Gist-Brocases N.V.), Bacillus lentus Alkaline Protease, and KAP (Bacillus alkalophilus subtilisin) from Kao.

At least one protease may be selected from serine proteases (EC 3.4.21). Serine proteases or serine peptidases (EC 3.4.21) are characterized by having a serine in the catalytically active site, which forms a covalent adduct with the substrate during the catalytic reaction. A serine protease may be selected from the group consisting of chymotrypsin (e.g., EC 3.4.21.1), elastase (e.g., EC 3.4.21.36), elastase (e.g., EC 3.4.21.37 or EC 3.4.21.71), granzyme (e.g., EC 3.4.21.78 or EC 3.4.21.79), kallikrein (e.g., EC 3.4.21.34, EC 3.4.21.35, EC 3.4.21.118, or EC 3.4.21.119) plasmin (e.g., EC 3.4.21.7), trypsin (e.g., EC 3.4.21.4), thrombin (e.g., EC 3.4.21.5) and subtilisin (also known as subtilopeptidase, e.g., EC 3.4.21.62), the latter hereinafter also being referred to as “subtilisin”.

### Cellulase

“Cellulases”, “cellulase enzymes” or “cellulolytic enzymes” are enzymes involved in hydrolysis of cellulose. Three major types of cellulases are known, namely endo-ss-1,4-glucanase (endo-1,4-P-D-glucan 4-glucanohydrolase, E.C. 3.2.1.4; hydrolyzing β-1,4-glucosidic bonds in cellulose), cellobiohydrolase (1,4-P-D-glucan cellobiohydrolase, EC 3.2.1.91), and ss-glucosidase (EC 3.2.1.21).

Cellulase enzymes have been described in patents and published patent applications including, but not limited to: WO1997/025417, WO1998/024799, WO2003/068910, WO2005/003319, and WO2009020459.

Commercially available cellulase enzymes include are Celluzyme™, Endolase™ Carezyme™, Cellusoft™, Renozyme™, Celluclean™ (from Novozymes A/S), Ecostone™, Biotouch™, Econase™, Ecopulp™ (from AB Enzymes Finland), Clazinase™, and Puradax HA™, Genencor detergent cellulase L, IndiAge™ Neutra (from Genencor International Inc./DuPont), Revitalenz™ (2000 from DuPont), Primafast™ (DuPont) and KAC-500™ (from Kao Corporation).

Cellulases according to the invention have “cellulolytic activity” or “cellulase activity”. Assays for measurement of cellulolytic activity are known to those skilled in the art. For example, cellulolytic activity may be determined by cellulase hydrolyses carboxymethyl cellulose to reducing carbohydrates, the reducing ability of which is determined colorimetrically by means of the ferricyanide reaction, according to Hoffman, W. S., J. Biol. Chem. 120, 51 (1937).

### Mannanase

Mannanase (E.C. 3.2.1.78) enzymes hydrolyze internal β-1,4 beta-D-mannosidic linkages in mannans, galactomannans and glucomannans. “Mannanase” may be an alkaline mannanase of Family 5 or 26. Mannanase are useful components of washing and/or cleaning formulations since mannanase remove part of hemicellulose containing stains. Insufficient removal of these types of stains may e.g. result in fabric graying. The major constituents of hemicellulose are hetero-1,4-D-xylans and herto-1,4-beta-mannans. Mannans are polysaccharides with a backbone of β-1,4-linked D-mannopyranosyl residues, which can contain galactose or acetyl substitutions and may have glucose residues in the backbone. Mannanase enzymes are known to be derived from wild-type from Bacillus or Humicola, particularly B. agaradhaerens, B. licheniformis, B. halodurans, B. clausii, or H. insolens. Suitable mannanases are described in WO 99/064619.

Commercially available mannanase enzymes include: Mannaway® (Novozymes AIS).

### Pectate Lyase

Pectate lyase (E.C. 4.2.2.2) enzymes eliminative cleavage of (1->4)-alpha-D-galacturonan to give oligosaccharides with 4-deoxy-alpha-D-galact-4-enuronosyl groups at their non-reducing ends.

Pectate lyase enzymes have been described in patents and published patent applications including, but not limited to: WO2004/090099. Pectate lyase are known to be derived from Bacillus, particularly B. licheniformis or B. agaradhaerens, or a variant derived of any of these, e.g. as described in U.S. Pat. No. 6,124,127, WO 99/027083, WO 99/027084, WO 2002/006442, WO 2002/092741, WO 2003/095638.

Commercially available pectate lyase enzymes include: Xpect™, Pectawash™ and Pectaway™ (Novozymes A/S); PrimaGreen™, EcoScour (DuPont).

### Nuclease

Nuclease (EC 3.1.21.1) also known as Deoxyribonuclease I, or DNase preforms endonucleolytic cleavage to 5′-phosphodinucleotide and 5′-phosphooligonucleotide end-products.

Nuclease enzymes have been described in patents and published patent applications including, but not limited to: U.S. Pat. No. 3,451,935, GB1300596, DE10304331, WO2015155350, WO2015155351, WO2015166075, WO2015181287, and WO2015181286.

In one aspect of the invention, at least one amylase variant of the invention is provided in combination with at least one protease. In one embodiment, an amylase variant of the invention is stable in the presence of at least one protease. In one embodiment, an amylase variant of the invention has increased protease stability when compared to the respective amylase parent. In one embodiment, at least one protease is selected from subtilisin 309 as disclosed as sequence a) in Table I of WO 89/06279 or a variant thereof which is at least 80% identical thereto and has proteolytic activity. In one embodiment, an amylase variant of the invention has increased protease stability in the presence of said subtilisin 309 or a variant thereof which is at least 80% identical thereto when compared to the amylase according to SEQ ID NO: 101.

The protease may itself be stabilized by a protease stabilizer or the protease may be non-stabilized. In one embodiment, an amylase variant of the invention has increased protease stability in the presence of a non-stabilized subtilisin 309 or a non-stabilized variant thereof which is at least 80% identical thereto, when compared to the amylase according to SEQ ID NO: 101.

### Method of Making

In another embodiment, the present invention refers to a method of making the variant polypeptide as described herein, comprising: providing a nucleic acid sequence encoding the polypeptide described herein, transforming the nucleic acid sequence into a host cell, cultivating the host cell to produce the variant polypeptide, and optionally purifying the variant polypeptide from the host cell.

A polynucleotide encoding a polypeptide may be “expressed”. The term “expression” or “gene expression” means the transcription of a specific gene or specific genes or specific nucleic acid construct. The term “expression” or “gene expression” means the transcription of a gene or genes or genetic construct into structural RNA (e.g., rRNA, tRNA) or mRNA with or without subsequent translation of the latter into a protein. The process includes transcription of DNA and processing of the resulting mRNA product.

Industrial production of enzymes usually is done by using expression systems. “Expression system” may mean a host microorganism, expression hosts, host cell, production organism, or production strain and each of these terms can be used interchangeably. In one embodiment, the expression host is selected from the group consisting of: a bacterial expression system, a yeast expression system, a fungal expression system, and a synthetic expression system. The expression host may be a wildtype cell or a recombinant cell. “Wild-type cells” herein means cells prior to a certain modification. The term “recombinant cell” (also called “genetically modified cell” herein) refers to a cell which has been genetically altered, modified or engineered such it that exhibits an altered, modified or different genotype as compared to the wild-type cell which it was derived from. The “recombinant cell” may comprise an exogenous polynucleotide encoding a certain protein or enzyme and therefore may express said protein or enzyme.

Thus, in one embodiment, the invention is directed to a genetic construct comprising a polynucleotide encoding the amylase as described herein.

In one embodiment, the invention is directed to a host cell comprising a polynucleotide encoding the amylase as described herein.

In yet another embodiment, the present invention is directed to a method of expressing a polynucleotide, comprising the steps of

(a) providing a host cell comprising a heterologous nucleic acid construct comprising a polynucleotide encoding the amylase described herein by introducing the nucleic acid construct comprising the polynucleotide encoding the amylase as described herein into the host cell;
(b) cultivating the recombinant host cell of step (a) under conditions conductive for the expression of the polynucleotide; and
(c) optionally, recovering a protein of interest encoded by the polynucleotide.

Examples of expression systems include but are not limited to: Aspergillus niger, Aspergillus oryzae, Hansenula polymorpha, Thermomyces lanuginosus, Fusarium oxysporum, Fusarium heterosporum, Escherichia coli, Bacillus, preferably Bacillus subtilis, or Bacillus licheniformis, Pseudomonas, preferably Pseudomonas fluorescens, Pichia pastoris (also known as Komagataella phaffin, Myceliopthora thermophile (C1), Themothelomyces thermophila, Schizosaccharomyces pombe, Trichoderma, preferably Trichoderma reesei and Saccharomyces, preferably Saccharomyces cerevisiae. The variant polypeptides may be produced using the expression system listed above.

In one embodiment, the bacterial expression system is selected from an E. coli, a Bacillus, a Pseudomonas, and a Streptomyces. In one embodiment, the yeast expression system is selected from a Candida, a Pichia, a Saccharomyces, and/or a Schizosaccharomyces. In one embodiment, the fungal expression system is selected from a Penicillium, an Aspergillus, a Fusarium, a Myceliopthora, a Rhizomucor, a Rhizopus, a Thermomyces, and a Trichoderma.

The term “heterologous” (or exogenous or foreign or recombinant) in the context of polynucleotides and polypeptides is defined herein as:

(a) not native to the host cell;

(b) native to the host cell but structural modifications, e.g., deletions, substitutions, and/or insertions, are included as a result of manipulation of the DNA of the host cell by recombinant DNA techniques to alter the native sequence; or

(c) native to the host cell but expression is quantitatively altered, or expression is directed from a genomic location different from the native host cell as a result of manipulation of the DNA of the host cell by recombinant DNA techniques, e.g., a stronger promoter.

With respect to two or more polynucleotide sequences or two or more amino acid sequences, the term “heterologous” is used to characterize that the two or more polynucleotide sequences or two or more amino acid sequences do not occur naturally in the specific combination with each other.

“Genetic Construct” or “expression cassette” as used herein, is a DNA molecule composed of at least one sequence of interest to be expressed, operably linked to one or more control sequences (at least to a promoter) as described herein. Typically, the expression cassette comprises three elements: a promoter sequence, an open reading frame, and a 3′ untranslated region that, in eukaryotes, usually contains a polyadenylation site. Additional regulatory elements may include transcriptional as well as translational enhancers. An intron sequence may also be added to the 5′ untranslated region (UTR) or in the coding sequence to increase the amount of the mature message that accumulates in the cytosol. The expression cassette may be part of a vector or may be integrated into the genome of a host cell and replicated together with the genome of its host cell. The expression cassette usually is capable of increasing or decreasing expression.

The term “vector” as used herein comprises any kind of construct suitable to carry foreign polynucleotide sequences for transfer to another cell, or for stable or transient expression within a given cell. The term “vector” as used herein encompasses any kind of cloning vehicles, such as but not limited to plasmids, phagemids, viral vectors (e.g., phages), bacteriophage, baculoviruses, cosmids, fosmids, artificial chromosomes, or and any other vectors specific for specific hosts of interest. Low copy number or high copy number vectors are also included. Foreign polynucleotide sequences usually comprise a coding sequence which may be referred to herein as “gene of interest”. The gene of interest may comprise introns and exons, depending on the kind of origin or destination of host cell.

A vector as used herein may provide segments for transcription and translation of a foreign polynucleotide upon transformation into a host cell or host cell organelles. Such additional segments may include regulatory nucleotide sequences, one or more origins of replication that is required for its maintenance and/or replication in a specific cell type, one or more selectable markers, a polyadenylation signal, a suitable site for the insertion of foreign coding sequences such as a multiple cloning site etc. One example is when a vector is required to be maintained in a bacterial cell as an episomal genetic element (e.g. plasmid or cosmid molecule). Non-limiting examples of suitable origins of replication include the f1-ori and colE1.

A vector may replicate without integrating into the genome of a host cell, e.g. as a plasmid in a bacterial host cell, or it may integrate part or all of its DNA into the genome of the host cell and thus lead to replication and expression of its DNA.

Foreign nucleic acid may be introduced into a vector by means of cloning. Cloning may mean that by cleavage of the vector (e.g. within the multiple cloning site) and the foreign polynucleotide by suitable means and methods (e.g., restriction enzymes), fitting structures within the individual nucleic acids may be created that enable the controlled fusion of said foreign nucleic acid and the vector.

Once introduced into the vector, the foreign nucleic acid comprising a coding sequence may be suitable to be introduced (transformed, transduced, transfected, etc.) into a host cell or host cell organelles. A cloning vector may be chosen suitable for expression of the foreign polynucleotide sequence in the host cell or host cell organelles.

The term “introduction” or “transformation” as referred to herein encompasses the transfer of an exogenous polynucleotide into a host cell, irrespective of the method used for transfer. That is, the term “transformation” as used herein is independent from vector, shuttle system, or host cell, and it not only relates to the polynucleotide transfer method of transformation as known in the art (cf., for example, Sambrook, J. et al. (1989) Molecular Cloning: A Laboratory Manual, 2nd Ed., Cold Spring Harbor Laboratory Press, Cold Spring Harbor, N.Y.), but it encompasses any further kind polynucleotide transfer methods such as, but not limited to, transduction or transfection. Plant tissue capable of subsequent clonal propagation, whether by organogenesis or embryogenesis, may be transformed with a genetic construct and a whole plant regenerated therefrom. The particular tissue chosen will vary depending on the clonal propagation systems available for, and best suited to, the particular species being transformed.

In one embodiment of the invention, a vector is used for transformation of a host cell.

The polynucleotide may be transiently or stably introduced into a host cell and may be maintained non-integrated, for example, as a plasmid. “Stable transformation” may mean that the transformed cell or cell organelle passes the nucleic acid comprising the foreign coding sequence on to the next generations of the cell or cell organelles. Usually stable transformation is due to integration of nucleic acid comprising a foreign coding sequence into the chromosomes or as an episome (separate piece of nuclear DNA).

“Transient transformation” may mean that the cell or cell organelle once transformed expresses the foreign nucleic acid sequence for a certain time—mostly within one generation. Usually transient transformation is due to nucleic acid comprising a foreign nucleic acid sequence is not integrated into the chromosomes or as an episome.

Alternatively, it may be integrated into the host genome. The resulting transformed plant cell may then be used to regenerate a transformed plant in a manner known to persons skilled in the art.

Recombinant cells may exhibit “increased” or “decreased” expression when compared to the respective wild-type cell.

The term “increased expression”, “enhanced expression” or “overexpression” as used herein means any form of expression that is additional to the original wild-type expression level (which can be absence of expression or immeasurable expression as well). Reference herein to “increased expression”, “enhanced expression” or “overexpression” is taken to mean an increase in gene expression and/or, as far as referring to polypeptides, increased polypeptide levels and/or increased polypeptide activity, relative to control organisms. The increase in expression may be in increasing order of preference at least 5%, at least 10%, at least 20%, at least 30%, at least 40%, at least 50%, at least 60%, at least 70%, at least 80%, at least 85%, at least 90%, or 100% or even more compared to that of control organisms.

Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to increase expression of a nucleic acid encoding the polypeptide of interest. For example, endogenous promoters may be altered in vivo by mutation, deletion, and/or substitution (see, Kmiec, U.S. Pat. No. 5,565,350; Zarling et al., WO 93/22443), or isolated promoters may be introduced into an organism in the proper orientation and distance from a gene of the present invention so as to control the expression of the gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in expression constructs has been shown to increase gene expression at both the mRNA and protein levels up to 1000-fold (Buchman and Berg (1988) Mol. Cell biol. 8: 4395-4405; Callis et al. (1987) Genes Dev 1:1183-1200). Such intron enhancement of gene expression is typically greatest when placed near the 5′ end of the transcription unit.

To obtain increased expression or overexpression of a polypeptide most commonly the nucleic acid encoding this polypeptide is overexpressed in sense orientation with a polyadenylation signal. Introns or other enhancing elements may be used in addition to a promoter suitable for driving expression with the intended expression pattern.

Enzymes are generally produced commercially by using recombinant cells which express the desired enzyme by cultivation of the same under conditions suitable for expression of the desired enzyme.

Cultivation normally takes place in a suitable nutrient medium allowing the recombinant cells to grow (this process may be called fermentation) and express the desired protein. At the end of fermentation, fermentation broth is collected and may be further processed, wherein the fermentation broth comprises a liquid fraction and a solid fraction.

The enzyme of interest may be further purified from the fermentation broth. The term “purification” or “purifying” refers to a process in which at least one component, e.g., a protein of interest, is separated from at least another component, e.g., a particulate matter of a fermentation broth, and transferred into a different compartment or phase, wherein the different compartments or phases do not necessarily need to be separated by a physical barrier. Examples of such different compartments are two compartments separated by a filtration membrane or cloth, i.e., filtrate and retentate; examples of such different phases are pellet and supernatant or cake and filtrate, respectively. The resulting solution after purifying the enzyme of interest from the fermentation broth is called herein “purified enzyme solution”.

The desired enzyme may be secreted (into the liquid fraction of the fermentation broth) or may not be secreted from the host cells (and therefore is comprised in the cells of the fermentation broth). Depending on this, the desired enzyme may be recovered from the liquid fraction of the fermentation broth or from cell lysates. Recovery of the desired enzyme uses methods known to those skilled in the art. Suitable methods for recovery of proteins or enzymes from fermentation broth include but are not limited to collection, centrifugation, filtration, extraction, and precipitation. If the enzyme of interest precipitates or crystallizes in the fermentation broth or binds at least in part to the particulate matter of the fermentation broth additional treatment steps might be needed to release the enzyme from the biomass or solubilize enzyme crystals and precipitates. U.S. Pat. No. 6,316,240B1 describes a method for recovering an enzyme, which precipitates and/or crystallizes during fermentation, from the fermentation broth. In case the desired enzyme is comprised in the cells of the fermentation broth release of the enzyme from the cells might be needed. Release from the cells can be achieved for instance, but not being limited thereto, by cell lysis with techniques well known to the skilled person.

The purified enzyme solution may be further processed to form an “enzyme formulation”. “Enzyme formulation” means any non-complex formulation comprising a small number of ingredients, wherein the ingredients serve the purpose of stabilizing the enzymes comprised in the enzyme formulation and/or the stabilization of the enzyme formulation itself. The term “enzyme stability” relates to the retention of enzymatic activity as a function of time during storage or operation. The term “enzyme formulation stability” relates to the maintenance of physical appearance of the enzyme formulation during storage or operation as well as the avoidance of microbial contamination during storage or operation.

An “enzyme formulation” is a composition which is meant to be formulated into a complex formulation which itself may be determined for final use. An “enzyme formulation” according to the invention is not a complex formulation comprising several components, wherein the components are formulated into the complex formulation to exert each individually a specific action in a final application. A complex formulation may be without being limited thereto a detergent formulation, wherein individual detergent components are formulated in amounts effective in the washing performance of the detergent formulation.

In one aspect of the invention, at least one amylase variant of the invention is comprised in an enzyme formulation.

The enzyme formulation can be either solid or liquid. Enzyme formulations can be obtained by using techniques known in the art. For instance, without being limited thereto, solid enzyme formulations can be obtained by extrusion or granulation. Suitable extrusion and granulation techniques are known in the art and are described for instance in WO9419444A1 and WO9743482A1.

“Liquid” in the context of enzyme formulation is related to the physical appearance at 20° C. and 101.3 kPa.

Liquid enzyme formulations may comprise amounts of enzyme in the range of 0.1% to 40% by weight, or 0.5% to 30% by weight, or 1% to 25% by weight, or 3% to 10%, all relative to the total weight of the enzyme formulation.

The liquid enzyme formulation may comprise more than one type of enzyme. In one embodiment, the enzyme formulation comprises one or more amylases according to the present invention. In one embodiment, the enzyme formulation comprises one or more amylases according to the present invention and at least one additional enzyme selected from the group consisting of selected from the group consisting of: a second amylase, a lipase, a protease, a cellulase, a laccase, a pectinase, a nuclease, and any combination thereof.

Aqueous enzyme formulations of the invention may comprise water in amounts of more than about 50% by weight, more than about 60% by weight, more than about 70% by weight, or more than about 80% by weight, all relative to the total weight of the enzyme formulation.

Liquid enzyme formulations of the invention may comprise residual components such as salts originating from the fermentation medium, cell debris originating from the production host cells, metabolites produced by the production host cells during fermentation.

In one embodiment, residual components may be comprised in liquid enzyme formulations in amounts less than 30% by weight, less than 20% by weight less, than 10% by weight, or less than 5% by weight, all relative to the total weight of the aqueous enzyme formulation. In one embodiment, the enzyme formulation, in particular the liquid enzyme formulation, comprises in addition to the one or more enzymes one or more additional compounds selected from the group consisting of solvent, salt, pH regulator, preservative, stabilizer, chelators, and thickening agent. The preservative in a liquid enzyme formulation maybe a sorbitol, a benzoate, a proxel, or any combination therefore. The stabilizers in a liquid enzyme formulation maybe an MPG, a glycerol, an acetate, or any combination thereof. The chelators in a liquid enzyme formulation maybe a citrate.

In one embodiment, an enzyme formulation comprises at least one polypeptide variant of the invention and at least one preservative. Non-limiting examples of suitable preservatives include (quaternary) ammonium compounds, isothiazolinones, organic acids, and formaldehyde releasing agents. Non-limiting examples of suitable (quaternary) ammonium compounds include benzalkonium chlorides, polyhexamethylene biguanide (PHMB), Didecyldimethylammonium chloride (DDAC), and N-(3-aminopropyl)-N-dodecylpropane-1,3-diamine (Diamine). Non-limiting examples of suitable isothiazolinones include 1,2-benzisothiazolin-3-one (BIT), 2-methyl-2H-isothiazol-3-one (MIT), 5-chloro-2-methyl-2H-isothiazol-3-one (CIT), 2-octyl-2H-isothiazol-3-one (OIT), and 2-butyl-benzo[d]isothiazol-3-one (BBIT). Non-limiting examples of suitable organic acids include benzoic acid, sorbic acid, L-(+)-lactic acid, formic acid, and salicylic acid. Non-limiting examples of suitable formaldehyde releasing agent include N,N′-methylenebismorpholine (MBM), 2,2′,2″-(hexahydro-1,3,5-triazine-1,3,5-triyl)triethanol (HHT), (ethylenedioxy)dimethanol, .alpha.,.alpha.′,.alpha.″-trimethyl-1,3,5-triazine-1,3,5(2H,4H,6H)-triethanol (HPT), 3,3′-methylenebis[5-methyloxazolidine] (MBO), and cis-1-(3-chloroallyl)-3,5,7-triaza-1-azoniaadamantane chloride (CTAC).

Further useful preservatives include iodopropynyl butylcarbamate (IPBC), halogen releasing compounds such as dichloro-dimethyl-hydantoine (DCDMH), bromo-chloro-dimethyl-hydantoine (BCDMH), and dibromo-dimethyl-hydantoine (DBDMH); bromo-nitro compounds such as Bronopol (2-bromo-2-nitropropane-1,3-diol), 2,2-dibromo-2-cyanoacetamide (DBNPA); aldehydes such as glutaraldehyde; phenoxyethanol; Biphenyl-2-ol; and zinc or sodium pyrithione.

In one embodiment, an enzyme formulation comprises at least one polypeptide variant of the invention and at least one enzyme stabilizer. An enzyme stabilizer is selected from substances which are capable of reducing loss of enzymatic activity during storage of at least one enzyme comprised in a liquid enzyme formulation. Reduced loss of enzymatic activity within this invention may mean that the loss of enzymatic activity is reduced by at least 5%, by at least 10%, by at least 15%, by at least 20%, by at least 25%, by at least 30%, by at least 40%, by at least 50%, by least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% when compared to the initial enzymatic activity before storage. Preferred stabilizers are selected from the group consisting of salt (e.g., CaCl2), propanediol, polyethylene glycol, an MPG, a glycerol, an acetate, or any combination thereof.

### Enzyme Applications

In another embodiment, the polypeptide variant as described herein may be used in foods, for example the enzyme can be an additive for baking. The enzymes can be used in feed, for example the enzyme is an animal feed additive. The enzyme can be used in the starch processing industry, for example the amylases are used in the conversion of starch to ethanol or sugars (high fructose corn syrup) and other byproducts such as oil, dry distiller's grains, etc. The polypeptide variants are used in in pulp and paper processing, for example, the enzymes can be used for improving paper strength. The enzymes can be used for mining and oil well services, for example cellulases can be used for breaking guar during oil well fracturing. In one embodiment, the polypeptide variant as described herein are used in detergent formulations or cleaning formulations.

In one embodiment, the present invention refers to a method of preparing a dough or a baked product prepared from the dough, the method comprising adding one of the variant polypeptides having amylase activity as described herein to the dough and baking it. In one embodiment, the present invention refers to a method of use of the variant polypeptide having amylase activity as described herein for processing starch. In one embodiment, the present invention refers to a method of use of the variant polypeptide having amylase activity as described herein for cleaning or washing textiles, hard surfaces, or dishes. In one embodiment, the present invention refers to a method of use of the variant polypeptide having amylase activity as described herein for making ethanol. In one embodiment, the present invention refers to a method of use of the variant polypeptide having amylase activity as described herein for processing pulp or paper. In one embodiment, the present invention refers to a method of use of the variant polypeptide having amylase activity as described herein for feeding an animal.

In one embodiment, the amylases of the present invention are used in detergent formulations or cleaning formulations.

“Detergent formulation” or “cleaning formulation” means compositions designated for cleaning soiled material. Cleaning includes laundering and hard surface cleaning. Soiled material according to the invention includes textiles and/or hard surfaces.

The term “laundering” relates to both household laundering and industrial laundering and means the process of treating textiles with a solution containing a detergent composition of the present invention. The laundering process may be carried out by using technical devices such as a household or an industrial washing machine. Alternatively, the laundering process may be done by hand.

The term “textile” means any textile material including yarns (thread made of natural or synthetic fibers used for knitting or weaving), yarn intermediates, fibers, non-woven materials, natural materials, synthetic materials, as well as fabrics (a textile made by weaving, knitting or felting fibers) made of these materials such as garments (any article of clothing made of textile), cloths and other articles.

The term “fibers” includes natural fibers, synthetic fibers, and mixtures thereof. Examples of natural fibers are of plant (such as flax, jute and cotton) or animal origin, comprising proteins like collagen, keratin and fibroin (e.g. silk, sheep wool, angora, mohair, cashmere). Examples for fibers of synthetic origin are polyurethane fibers such as Spandex® or Lycra®, polyester fibers, polyolefins such as elastofin, or polyamide fibers such as nylon. Fibers may be single fibers or parts of textiles such as knitwear, woven, or nonwovens.

The term “hard surface cleaning” is defined herein as cleaning of hard surfaces wherein hard surfaces may include any hard surfaces in the household, such as floors, furnishing, walls, sanitary ceramics, glass, metallic surfaces including cutlery or dishes.

The term “dish wash” refers to all forms of washing dishes, e.g. by hand or automatic dish wash. Dish washing includes, but is not limited to, the cleaning of all forms of crockery such as plates, cups, glasses, bowls, all forms of cutlery such as spoons, knives, forks and serving utensils as well as ceramics, plastics such as melamine, metals, china, glass and acrylics.

The detergent formulation of the invention comprises one or more detergent component(s). The component(s) chosen depend(s) on the desired cleaning application and/or physical form of a detergent composition.

The term “detergent component” is defined herein to mean any types of ingredient, which is suitable for detergent compositions, such as surfactants, building agents, polymers, bleaching systems. Any component(s) known in the art acknowledging their known characteristics are suitable detergent component(s) according to the invention. Detergent components in one embodiment means components which provide washing or cleaning performance, or which effectively aid the processing (maintain physical characteristics during processing, storage and use; e.g. rheology modifiers, hydrotropes, desiccants) when present in effective amounts.

Usually, a detergent composition is a complex formulation of more than two detergent components.

Detergent components may have more than one function in the final application of a detergent formulation, therefore any detergent component mentioned in the context of a specific function herein, may also have another function in the final application of a detergent formulation. The function of a specific detergent component in the final application of a detergent formulation usually depends on its amount within the detergent formulation, i.e. the effective amount of a detergent component.

The term “effective amount” includes amounts of certain components to provide effective stain removal and effective cleaning conditions (e.g. pH, quantity of foaming), amounts of certain components to effectively provide optical benefits (e.g. optical brightening, dye transfer inhibition), and amounts of certain components to effectively aid the processing (maintain physical characteristics during processing, storage and use; e.g. rheology modifiers, hydrotropes, desiccants).

In one embodiment, a detergent formulation is a formulation of more than two detergent components, wherein at least one component is effective in stain-removal, at least one component is effective in providing the optimal cleaning conditions, and at least one component is effective in maintaining the physical characteristics of the detergent.

Cleaning performance is evaluated under relevant cleaning conditions. The term “relevant cleaning conditions” herein refers to the conditions, particularly cleaning temperature, time, cleaning mechanics, suds concentration, type of detergent and water hardness, actually used in laundry machines, automatic dish washers or in manual cleaning processes.

Individual detergent components and usage in detergent compositions are known to those skilled in the art. Suitable detergent components comprise inter alia surfactants, builders, polymers, alkaline, bleaching systems, fluorescent whitening agents, suds suppressors and stabilizers, hydrotropes, and corrosion inhibitors. Further examples are described e.g. in “complete Technology Book on Detergents with Formulations (Detergent Cake, Dishwashing Detergents, Liquid & Paste Detergents, Enzyme Detergents, Cleaning Powder & Spray Dried Washing Powder)”, Engineers India Research Institute (EIRI), 6^{th} edition (2015). Another reference book for those skilled in the art may be “Detergent Formulations Encyclopedia”, Solverchem Publications, 2016.

Detergent components vary in type and/or amount in a detergent formulation depending on the desired application such as laundering white textiles, colored textiles, and wool. The component(s) chosen further depend(s) on physical form of a detergent formulation (liquid, solid, gel, provided in pouches or as a tablet, etc.). The component(s) chosen e.g. for laundering formulations further depend on regional conventions which themselves are related to aspects like washing temperatures used, mechanics of laundry machine (vertical vs. horizontal axis machines), water consumption per wash cycle etc. and geographical characteristics like average hardness of water.

For example: A low detergent concentration system includes laundering formulations where less than about 800 ppm of detergent components are present in the wash water. A medium detergent concentration includes laundering formulations where between about 800 ppm and about 2,000 ppm of detergent components are present in the wash water. A high detergent concentration includes laundering formulations where more than about 2,000 ppm of detergent components are present in the wash water.

The numeric ranges recited for the individual detergent components provide amounts comprised in detergent compositions. Such ranges have to be understood to be inclusive of the numbers defining the range and include each integer within the defined range.

If not described otherwise, “% by weight” or “% w/w” is meant to be related to total detergent composition. In this case “% by weight” or “% w/w” is calculated as follows: concentration of a substance as the weight of that substance divided by the total weight of the composition, multiplied by 100.

Detergent formulations of the invention may comprise one or more surfactant(s). “Surfactant” (synonymously used herein with “surface active agent”) means an organic chemical that, when added to a liquid, changes the properties of that liquid at an interface. According to its ionic charge, a surfactant is called non-ionic, anionic, cationic, or amphoteric.

Non-limiting examples of surfactants are disclosed McCutcheon's 2016 Detergents and Emulsifiers, and McCutcheon's 2016 Functional Materials, both North American and International Edition, MC Publishing Co, 2016 edition. Further useful examples are disclosed in earlier editions of the same publications which are known to those skilled in the art.

Non-ionic surfactant means a surfactant that contains neither positively nor negatively charged (i.e. ionic) functional groups. In contrast to anionic and cationic surfactants, non-ionic surfactants do not ionize in solution.

### Example 1: Amylase Fragment Identification and Reassembly

The parent amino acid sequences of the amylases (Amylase 51, SEQ ID NO: 101) and SEQ ID NO: 131, were aligned in silico and divided into 4 regions spanning the aligned genes. Each of the four regions, (hereafter referred to as Region 1, Region 2, Region 3, and Region 4) was further divided into fragments as described in the table below. For example, Region 1 has fragment “a” (SEQ ID NO:103) or fragment “b” (SEQ ID NO:104). Altogether 28 unique fragments were designed having the amino acid sequences SEQ ID NO: 103 to 130. The fragments were synthetically generated by Integrated DNA Technologies, Inc. (IDT) (Illinois, USA) and reassembled in an ordered fashion to produce different combinations of fragments resulting in new hybrid amylase backbones (SEQ ID NOs:1-100) that have different amino acid sequences when compared to the parent backbones. Each new hybrid amylase backbone was comprised of a single fragment from each of Region 1, Region 2, Region 3, and Region 4, respectively, from the beginning to the end of the mature gene coding sequence. The fragments were assembled using a sequential Golden Gate assembly approach as follows: Each of the 28 gene fragments were designed with flanking regions containing TypeIIs restriction enzyme recognition sites and specific nucleotides to generate compatible overhangs with the relevant adjacent fragments. All 28 fragments were PCR amplified and fragments from each region were pooled in an equimolar ratio. An E. coli vector backbone was then PCR amplified using primers to add terminal TypeIIs restriction enzyme recognition sites and specific nucleotides to create compatible overhangs for ligation with the 5′ termini of Region 1 and the 3′ terminus of Region4. Golden Gate assembly was used to ligate the E. coli vector backbone and the pooled fragments from Region 1, Region 2, Region 3, and Region 4. The assembled constructs were subsequently transformed into an E. coli cloning host and plated onto selective agar plates. The resulting colonies were mixed together and plasmid DNA from the mixture was extracted, resulting in a library of fully assembled hybrid amylases. PCR was then used to amplify the full-length hybrid amylase variants from the extracted plasmid library and add appropriate restriction recognition sites and Golden Gate compatible overhangs on the termini. Similarly, a Bacillus expression vector was PCR amplified using primers to add terminal Golden Gate compatible sequences. The amplified Bacillus backbone and amplified library of hybrid amylases were then assembled using a second Golden Gate reaction and transformed into a Bacillus subtilis host and grown on selective agar plates. The resulting colonies were screened for correctly assembled full-length hybrid amylases via colony PCR and Sanger sequencing.

| Full Length (Mature) | | | | | | |
|---|---|---|---|---|---|---|
| Hybrid Amylase | | | | | | |
| | Amino | | | | | |
| | Acid | DNA | | | | |
| | Sequence | Sequence | | | | |
| Enzyme | (SEQ ID | (SEQ ID | Regions and fragments Amino Acid Sequence (SEQ ID NO:) | | | |
| Name | NO:) | NO:) | Region 1 | Region 2 | Region 3 | Region 4 |
| Amylase_01 | 1 | 2 | b is SEQ ID NO: 104 | f is SEQ ID NO: 110 | f is SEQ ID NO: 118 | e is SEQ ID NO: 125 |
| Amylase_02 | 3 | 4 | b is SEQ ID NO: 104 | b is SEQ ID NO: 106 | h is SEQ ID NO: 120 | f is SEQ ID NO: 126 |
| Amylase_03 | 5 | 6 | b is SEQ ID NO: 104 | b is SEQ ID NO: 106 | f is SEQ ID NO: 118 | g is SEQ ID NO: 127 |
| Amylase_04 | 7 | 8 | b is SEQ ID NO: 104 | d is SEQ ID NO: 108 | f is SEQ ID NO: 118 | f is SEQ ID NO: 126 |
| Amylase_05 | 9 | 10 | a is SEQ ID NO: 103 | g is SEQ ID NO: 111 | e is SEQ ID NO: 117 | h is SEQ ID NO: 128 |
| Amylase_06 | 11 | 12 | a is SEQ ID NO: 103 | h is SEQ ID NO: 112 | e is SEQ ID NO: 117 | g is SEQ ID NO: 127 |
| Amylase_07 | 13 | 14 | b is SEQ ID NO: 104 | h is SEQ ID NO: 112 | e is SEQ ID NO: 117 | b is SEQ ID NO: 122 |
| Amylase_08 | 15 | 16 | b is SEQ ID NO: 104 | d is SEQ ID NO: 108 | e is SEQ ID NO: 117 | f is SEQ ID NO: 126 |
| Amylase_09 | 17 | 18 | a is SEQ ID NO: 103 | h is SEQ ID NO: 112 | e is SEQ ID NO: 117 | f is SEQ ID NO: 126 |
| Amylase_10 | 19 | 20 | b is SEQ ID NO: 104 | d is SEQ ID NO: 108 | e is SEQ ID NO: 117 | d is SEQ ID NO: 124 |
| Amylase_11 | 21 | 22 | a is SEQ ID NO: 103 | d is SEQ ID NO: 108 | f is SEQ ID NO: 118 | g is SEQ ID NO: 127 |
| Amylase_12 | 23 | 24 | b is SEQ ID NO: 104 | b is SEQ ID NO: 106 | b is SEQ ID NO: 114 | e is SEQ ID NO: 125 |
| Amylase_13 | 25 | 26 | b is SEQ ID NO: 104 | d is SEQ ID NO: 108 | e is SEQ ID NO: 117 | e is SEQ ID NO: 125 |
| Amylase_14 | 27 | 28 | b is SEQ ID NO: 104 | f is SEQ ID NO: 110 | e is SEQ ID NO: 117 | a is SEQ ID NO: 121 |
| Amylase_15 | 29 | 30 | a is SEQ ID NO: 103 | b is SEQ ID NO: 106 | e is SEQ ID NO: 117 | h is SEQ ID NO: 128 |
| Amylase_16 | 31 | 32 | a is SEQ ID NO: 103 | h is SEQ ID NO: 112 | g is SEQ ID NO: 119 | b is SEQ ID NO: 122 |
| Amylase_17 | 33 | 34 | a is SEQ ID NO: 103 | h is SEQ ID NO: 112 | e is SEQ ID NO: 117 | e is SEQ ID NO: 125 |
| Amylase_18 | 35 | 36 | b is SEQ ID NO: 104 | b is SEQ ID NO: 106 | e is SEQ ID NO: 117 | e is SEQ ID NO: 125 |
| Amylase_19 | 37 | 38 | b is SEQ ID NO: 104 | e is SEQ ID NO: 109 | e is SEQ ID NO: 117 | a is SEQ ID NO: 121 |
| Amylase_20 | 39 | 40 | a is SEQ ID NO: 103 | f is SEQ ID NO: 110 | e is SEQ ID NO: 117 | e is SEQ ID NO: 125 |
| Amylase_21 | 41 | 42 | a is SEQ ID NO: 103 | h is SEQ ID NO: 112 | e is SEQ ID NO: 117 | i is SEQ ID NO: 129 |
| Amylase_22 | 43 | 44 | b is SEQ ID NO: 104 | b is SEQ ID NO: 106 | e is SEQ ID NO: 117 | c is SEQ ID NO: 123 |
| Amylase_23 | 45 | 46 | a is SEQ ID NO: 103 | f is SEQ ID NO: 110 | e is SEQ ID NO: 117 | c is SEQ ID NO: 123 |
| Amylase_24 | 47 | 48 | a is SEQ ID NO: 103 | f is SEQ ID NO: 110 | c is SEQ ID NO: 115 | c is SEQ ID NO: 123 |
| Amylase_25 | 49 | 50 | a is SEQ ID NO: 103 | d is SEQ ID NO: 108 | e is SEQ ID NO: 117 | g is SEQ ID NO: 127 |
| Amylase_26 | 51 | 52 | a is SEQ ID NO: 103 | d is SEQ ID NO: 108 | f is SEQ ID NO: 118 | e is SEQ ID NO: 125 |
| Amylase_27 | 53 | 54 | b is SEQ ID NO: 104 | d is SEQ ID NO: 108 | e is SEQ ID NO: 117 | b is SEQ ID NO: 122 |
| Amylase_28 | SS | 56 | b is SEQ ID NO: 104 | d is SEQ ID NO: 108 | a is SEQ ID NO: 113 | c is SEQ ID NO: 123 |
| Amylase_29 | 57 | 58 | a is SEQ ID NO: 103 | d is SEQ ID NO: 108 | e is SEQ ID NO: 117 | f is SEQ ID NO: 126 |
| Amylase_30 | 59 | 60 | a is SEQ ID NO: 103 | f is SEQ ID NO: 110 | e is SEQ ID NO: 117 | b is SEQ ID NO: 122 |
| Amylase_31 | 61 | 62 | a is SEQ ID NO: 103 | b is SEQ ID NO: 106 | e is SEQ ID NO: 117 | f is SEQ ID NO: 126 |
| Amylase_32 | 63 | 64 | a is SEQ ID NO: 103 | f is SEQ ID NO: 110 | g is SEQ ID NO: 119 | a is SEQ ID NO: 121 |
| Amylase_33 | 65 | 66 | a is SEQ ID NO: 103 | d is SEQ ID NO: 108 | b is SEQ ID NO: 114 | e is SEQ ID NO: 125 |
| Amylase_34 | 67 | 68 | a is SEQ ID NO: 103 | d is SEQ ID NO: 108 | e is SEQ ID NO: 117 | d is SEQ ID NO: 124 |
| Amylase_35 | 69 | 70 | a is SEQ ID NO: 103 | d is SEQ ID NO: 108 | g is SEQ ID NO: 119 | c is SEQ ID NO: 123 |
| Amylase_36 | 71 | 72 | b is SEQ ID NO: 104 | d is SEQ ID NO: 108 | e is SEQ ID NO: 117 | a is SEQ ID NO: 121 |
| Amylase_37 | 73 | 74 | b is SEQ ID NO: 104 | b is SEQ ID NO: 106 | e is SEQ ID NO: 117 | a is SEQ ID NO: 121 |
| Amylase_38 | 75 | 76 | a is SEQ ID NO: 103 | f is SEQ ID NO: 110 | e is SEQ ID NO: 117 | a is SEQ ID NO: 121 |
| Amylase_39 | 77 | 78 | b is SEQ ID NO: 104 | c is SEQ ID NO: 107 | e is SEQ ID NO: 117 | a is SEQ ID NO: 121 |
| Amylase_40 | 79 | 80 | a is SEQ ID NO: 103 | d is SEQ ID NO: 108 | e is SEQ ID NO: 117 | e is SEQ ID NO: 125 |
| Amylase_41 | 81 | 82 | a is SEQ ID NO: 103 | b is SEQ ID NO: 106 | e is SEQ ID NO: 117 | e is SEQ ID NO: 125 |
| Amylase_42 | 83 | 84 | a is SEQ ID NO: 103 | d is SEQ ID NO: 108 | e is SEQ ID NO: 117 | j is SEQ ID NO: 130 |
| Amylase_43 | 85 | 86 | a is SEQ ID NO: 103 | b is SEQ ID NO: 106 | e is SEQ ID NO: 117 | c is SEQ ID NO: 123 |
| Amylase_44 | 87 | 88 | a is SEQ ID NO: 103 | e is SEQ ID NO: 109 | e is SEQ ID NO: 117 | a is SEQ ID NO: 121 |
| Amylase_45 | 89 | 90 | a is SEQ ID NO: 103 | d is SEQ ID NO: 108 | e is SEQ ID NO: 117 | b is SEQ ID NO: 122 |
| Amylase_46 | 91 | 92 | a is SEQ ID NO: 103 | c is SEQ ID NO: 107 | e is SEQ ID NO: 117 | c is SEQ ID NO: 123 |
| Amylase_47 | 93 | 94 | a is SEQ ID NO: 103 | b is SEQ ID NO: 106 | e is SEQ ID NO: 117 | b is SEQ ID NO: 122 |
| Amylase_48 | 95 | 96 | a is SEQ ID NO: 103 | d is SEQ ID NO: 108 | e is SEQ ID NO: 117 | a is SEQ ID NO: 121 |
| Amylase_49 | 97 | 98 | a is SEQ ID NO: 103 | b is SEQ ID NO: 106 | e is SEQ ID NO: 117 | a is SEQ ID NO: 121 |
| Amylase_50 | 99 | 100 | a is SEQ ID NO: 103 | b is SEQ ID NO: 106 | a is SEQ ID NO: 113 | a is SEQ ID NO: 121 |
| Amylase_51 | 101 | 102 | a is SEQ ID NO: 103 | a is SEQ ID NO: 105 | a is SEQ ID NO: 113 | a is SEQ ID NO: 121 |

### Example 2: Amylase Expression and Protein Quantification

Single colonies of the expression strains were picked into 60 μL of LB media containing 2% glucose and kanamycin in a 384-well plate. The cultures were grown at 37 degrees C. for 16 hours, then 15 μL of culture was used to inoculate 600 μL of 2 L media (twice the components of LB media) containing 7.5% maltose and 20 μg/mL kanamycin in a 96-well plate. The cultures were grown at 37 degrees C. for 48 hours, after which the supernatant was harvested by centrifugation at 5000×g for 15 minutes at 4 degrees C. The expression levels (concentration in mg/mL) of variant polypeptide hybrids having alpha-amylase activity were identified by LabChip (LC) analysis and corresponding expression plasmids were sequenced.

The results, which were normalized for expression as provided in the table below, show that many of the hybrid amylase enzymes have an increase in expression when compared to the parent enzyme (Amylase_051). For example, Amylase_01, Amylase_02, Amylase_03, Amylase_04, Amylase_05, Amylase_06, Amylase_07, Amylase_08, Amylase_09, Amylase_11, Amylase_12, Amylase_13, Amylase_14, Amylase_15, Amylase_17, Amylase_18, Amylase_19, Amylase_20, Amylase_21, Amylase_22, Amylase_23, Amylase_25, Amylase_26, Amylase_27, Amylase_28, Amylase_29, Amylase_30, Amylase_31, Amylase_33, Amylase_34, Amylase_36, Amylase_37, Amylase_38, Amylase_39, Amylase_40, Amylase_41, Amylase_42, Amylase_43, Amylase_44, Amylase_45, Amylase_46, Amylase_47, Amylase_48, Amylase_49, Amylase_50, have an increase in expression ranging from 1.01 to 1.27.

| Normalized | Enzyme |
|---|---|
| Expression | Name |
| 1.14 | Amylase_01 |
| 1.09 | Amylase_02 |
| 1.19 | Amylase_03 |
| 1.18 | Amylase_04 |
| 1.23 | Amylase_05 |
| 1.24 | Amylase_06 |
| 1.03 | Amylase_07 |
| 1.26 | Amylase_08 |
| 1.21 | Amylase_09 |
| 1.00 | Amylase_10 |
| 1.16 | Amylase_11 |
| 1.18 | Amylase_12 |
| 1.27 | Amylase_13 |
| 1.12 | Amylase_14 |
| 1.16 | Amylase_15 |
| 0.92 | Amylase_16 |
| 1.23 | Amylase_17 |
| 1.22 | Amylase_18 |
| 1.05 | Amylase_19 |
| 1.22 | Amylase_20 |
| 1.16 | Amylase_21 |
| 1.12 | Amylase_22 |
| 1.14 | Amylase_23 |
| 0.95 | Amylase_24 |
| 1.23 | Amylase_25 |
| 1.18 | Amylase_26 |
| 1.11 | Amylase_27 |
| 1.06 | Amylase_28 |
| 1.18 | Amylase_29 |
| 1.04 | Amylase_30 |
| 1.20 | Amylase_31 |
| 1.00 | Amylase_32 |
| 1.12 | Amylase_33 |
| 1.01 | Amylase_34 |
| 1.00 | Amylase_35 |
| 1.26 | Amylase_36 |
| 1.24 | Amylase_37 |
| 1.09 | Amylase_38 |
| 1.17 | Amylase_39 |
| 1.12 | Amylase_40 |
| 1.26 | Amylase_41 |
| 1.15 | Amylase_42 |
| 1.11 | Amylase_43 |
| 1.04 | Amylase_44 |
| 1.12 | Amylase_45 |
| 1.07 | Amylase_46 |
| 1.14 | Amylase_47 |
| 1.25 | Amylase_48 |
| 1.13 | Amylase_49 |
| 1.09 | Amylase_50 |
| 1.00 | Amylase_51 |

### Example 3: Red Starch and Residual Activity Assays

Quantitation of starch hydrolysis for the variant polypeptides containing alpha-amylase activity was measured using the Red Starch method as described by Megazyme, “Assay of Alpha-Amylase using Red-Starch” with the following modifications. 10 μL of 1.33% red starch prepared in 50 mM HEPES, pH 8.0 buffer was reacted with 10 μL enzyme diluted in 50 mM HEPES, pH 8.0 buffer at 25° C. The reaction was terminated after 10 minutes by the addition of 50 μL of 200 proof ethanol. After vigorous mixing the reaction was equilibrated for 10 minutes at room temperature followed by centrifugation at 1,200×g for 10 minutes. 40 of the reaction was transferred and the solution absorption (AU) was read at 510 nm in a BioTek plate reader. Residual activity was calculated by comparing the activity of each enzyme as measured using the red starch assay before and after a heat challenge. Heat challenges were conducted on enzyme diluted in 50 mM HEPES, pH 8.0 buffer in two separate instances with different heating regimes. In one instance, the diluted enzyme was heated to 80 degrees C. for 30 minutes, chilled at 8 degrees C. for 20 minutes, and equilibrated to room temperature for 20 minutes before being tested using the red starch assay at 25 degrees C. In a separate instance, the diluted enzyme was heated to 90 degrees C. for 15 minutes, chilled at 4 degrees C. for 10 minutes, then held at 24 degrees C. for 5 minutes before being tested using the red starch assay at 25 degrees C.

The table below shows the remaining activity of the amylase after heat treatment. The results, as provided in the table below, show that all the amylases are more thermostable when compared to the parent enzyme (Amylase_51).

Amylase Hybrid Residual Activity (90 degrees C.)

| Percent Activity | Enzyme |
|---|---|
| Remaining | Name |
| 50.2 | Amylase_01 |
| 71.5 | Amylase_02 |
| 96.3 | Amylase_03 |
| 54.1 | Amylase_04 |
| 51.1 | Amylase_05 |
| 65.7 | Amylase_06 |
| 51.9 | Amylase_07 |
| 53.0 | Amylase_08 |
| 50.7 | Amylase_09 |
| 50.2 | Amylase_10 |
| 116.1 | Amylase_11 |
| 72.5 | Amylase_12 |
| 60.4 | Amylase_13 |
| 63.5 | Amylase_14 |
| 71.0 | Amylase_15 |
| 65.7 | Amylase_16 |
| 59.2 | Amylase_17 |
| 56.9 | Amylase_18 |
| 61.9 | Amylase_19 |
| 54.0 | Amylase_20 |
| 52.3 | Amylase_21 |
| 51.7 | Amylase_22 |
| 93.5 | Amylase_23 |
| 76.0 | Amylase_24 |
| 51.0 | Amylase_25 |
| 64.5 | Amylase_26 |
| 56.9 | Amylase_27 |
| 82.4 | Amylase_28 |
| 51.9 | Amylase_29 |
| 51.0 | Amylase_30 |
| 51.4 | Amylase_31 |
| 63.4 | Amylase_32 |
| 53.7 | Amylase_33 |
| 65.5 | Amylase_34 |
| 56.6 | Amylase_35 |
| 55.9 | Amylase_36 |
| 50.3 | Amylase_37 |
| 121.0 | Amylase_38 |
| 62.7 | Amylase_39 |
| 61.6 | Amylase_40 |
| 67.3 | Amylase_41 |
| 73.0 | Amylase_42 |
| 70.7 | Amylase_43 |
| 83.9 | Amylase_44 |
| 63.5 | Amylase_45 |
| 65.5 | Amylase_46 |
| 120.4 | Amylase_47 |
| 89.3 | Amylase_48 |
| 59.2 | Amylase_49 |
| 63.4 | Amylase_50 |
| 11.1 | Amylase_51 |

### Example 4: Enzyme Stability in Detergent Formulations

The stability of the amylase enzymes in detergent was observed by adding the enzymes of interest into commercial detergent Persil Non-Bio. The detergents containing enzyme were then mixed and stored at 37° C. At day 0, 1, 2, 3, 7, and 14, an aliquot was taken from the detergents containing enzyme and measured for activity.

At each time point (day), amylase activity was measured by analyzing the rate of p-nitrophenol (pNP) released during the enzymatic hydrolysis of Infinity™ Amylase Liquid Stable Reagent. The sample was diluted in 50 mM MOPS, pH 7.0 prior to the assay. 100 μL of the diluted sample was added to 100 μL of Infinity™ Amylase Liquid Stable Reagent. The reaction was observed at room temperature using a BioTek plate reader at 405 nm. The activity remaining was calculated by comparing the MaxV at each time point to the initial value at day 0.

The results, as provided in the table below, show that many of the amylases are more stable (have a higher % activity remaining) in detergent formulations over time when compared to the parent enzyme Amylase_51. For example, Amylase_01, Amylase_02, Amylase_03, Amylase_04, Amylase_06, Amylase_07, Amylase_08, Amylase_10, Amylase_11, Amylase_14, Amylase_16, Amylase_17, Amylase_18, Amylase_21, Amylase_22, Amylase_23, Amylase_24, Amylase_26, Amylase_27, Amylase_28, Amylase_29, Amylase_30, Amylase_32, Amylase_34, Amylase_35, Amylase_36, Amylase_37, Amylase_38, Amylase_39, Amylase_40, Amylase_43, Amylase_44, Amylase_45, Amylase_47, Amylase_48, Amylase_49, and Amylase_50, have 0.8% to 21.6% activity remaining after 14 days in detergent formulations.

Enzyme stability (% activity remaining)
in detergent formulations over time

| 3 Days | 7 Days | 14 Days | Enzyme Name |
|---|---|---|---|
| 55.9 | 28.7 | 8.7 | Amylase_01 |
| 23.6 | 14.7 | 4.8 | Amylase_02 |
| 57.4 | 32.7 | 9.1 | Amylase_03 |
| 62.8 | 45.8 | 18.2 | Amylase_04 |
| 15.5 | 4.1 | 0 | Amylase_05 |
| 23.0 | 3.1 | 1.1 | Amylase_06 |
| 40.0 | 14.5 | 2.6 | Amylase_07 |
| 24.5 | 3.3 | 0.9 | Amylase_08 |
| 25.7 | 4.1 | 0 | Amylase_09 |
| 36.6 | 11.3 | 2.3 | Amylase_10 |
| 62.3 | 41.1 | 14.6 | Amylase_11 |
| 23.6 | 5.4 | 0 | Amylase_13 |
| 39.7 | 9.9 | 1.6 | Amylase_14 |
| 14.7 | 1.9 | 0 | Amylase_15 |
| 41.9 | 15.1 | 3.7 | Amylase_16 |
| 24.2 | 3.9 | 1.3 | Amylase_17 |
| 19.4 | 1.8 | 1.2 | Amylase_18 |
| 26.9 | 4.5 | 0 | Amylase_19 |
| 16.9 | 1.7 | 0 | Amylase_20 |
| 25.5 | 4.1 | 1.7 | Amylase_21 |
| 31.2 | 8.2 | 2.4 | Amylase_22 |
| 33.5 | 7.8 | 2.1 | Amylase_23 |
| 21.9 | 3.1 | 0 | Amylase_25 |
| 69.5 | 49.3 | 21.6 | Amylase_26 |
| 46.8 | 16.0 | 3.3 | Amylase_27 |
| 1.8 | 0 | 0 | Amylase_28 |
| 23.2 | 4.8 | 1.1 | Amylase_29 |
| 36.2 | 9.6 | 1.5 | Amylase_30 |
| 18.5 | 2.8 | 0 | Amylase_31 |
| 36.0 | 9.6 | 1.6 | Amylase_32 |
| 3.7 | 0 | 0 | Amylase_33 |
| 39.1 | 13.4 | 2.2 | Amylase_34 |
| 39.4 | 13.6 | 3.3 | Amylase_35 |
| 44.8 | 16.8 | 2.0 | Amylase_36 |
| 39.5 | 9.7 | 1.7 | Amylase_37 |
| 38.6 | 8.9 | 1.2 | Amylase_38 |
| 32.9 | 7.3 | 1.3 | Amylase_39 |
| 22.0 | 3.3 | 1.7 | Amylase_40 |
| 15.1 | 1.5 | 0 | Amylase_41 |
| 23.2 | 4.0 | 0 | Amylase_42 |
| 34.3 | 10.2 | 1.9 | Amylase_43 |
| 29.3 | 6.0 | 0.8 | Amylase_44 |
| 47.4 | 17.3 | 3.0 | Amylase_45 |
| 26.4 | 7.0 | 0 | Amylase_46 |
| 36.5 | 9.8 | 2.9 | Amylase_47 |
| 46.9 | 15.6 | 2.4 | Amylase_48 |
| 35.3 | 9.0 | 1.6 | Amylase_49 |
| 0.9 | 1.7 | 1.4 | Amylase_50 |
| 0.9 | 1.6 | 0 | Amylase_51 |

### Example 5: Enzyme Performance in Laundry Applications (Stain Removal)

Wash performance was measured for the hybrid amylases on cotton soiled with starch (EMPA161) purchased from CFT. Amylases were dosed at 0.1, 0.2, or 0.3 ppm in 10 mM HEPES pH 8.0, 2.7 mM hard water plus 5 g/L detergent ES1 (Lutensit A-LBS LAS 5,5% w/w, Edenor coco fatty acid C12-C18 coco fatty acid 2,4% w/w, Lutensol AO7 AEO 5,5% w/w, Texapon N70 FAEO 5,5% w/w, 1,2 propylene glycol 6,0% w/w, ethanol 2,0% w/w, KOH 2,2% w/w) plus 3% citrate, plus 150 mM CaCl2, in individual wells. Wash was conducted by agitating in orbital shakers (500 rpm) for 30 minutes at 40° C. Wash liquor was removed then the fabric was rinsed three times with water. The samples were dried overnight at room temperature. Wash performance was measured using digital image analysis of washed stains; the average RGB intensity was normalized to the parent for each sample; and then average of the three data points (0.1, 0.2, and 0.3 ppm) is provided.

The results, as provided in the table below, show the amylase enzymes that have improved performance (stain removal) when compared to the parent enzyme (Amylase_51). For example, Amylase_01, Amylase_02, Amylase_04, Amylase_05, Amylase_06, Amylase_08, Amylase_12, Amylase_13, Amylase_14, Amylase_18, Amylase_20, Amylase_21, Amylase_22, Amylase_23, Amylase_25, Amylase_26, Amylase_28, Amylase_31, Amylase_33, Amylase_36, Amylase_37, Amylase_38, Amylase_39, Amylase_41, Amylase_46, and Amylase_50, all have an Avg. RGB value from 1.07 to 1.68, which is an increase when compared to the parent enzyme (Amylase_051).

| RGB value for wash | |
|---|---|
| performance results | Enzyme |
| (Avg. RGB for all doses) | Name |
| 1.29 | Amylase_01 |
| 1.26 | Amylase_02 |
| 0.87 | Amylase_03 |
| 1.06 | Amylase_04 |
| 1.34 | Amylase_05 |
| 1.52 | Amylase_06 |
| 0.49 | Amylase_07 |
| 1.41 | Amylase_08 |
| 1.04 | Amylase_09 |
| 0.95 | Amylase_10 |
| 0.87 | Amylase_11 |
| 1.09 | Amylase_12 |
| 1.32 | Amylase_13 |
| 1.40 | Amylase_14 |
| 0.97 | Amylase_15 |
| 0.38 | Amylase_16 |
| 1.02 | Amylase_17 |
| 1.23 | Amylase_18 |
| 0.96 | Amylase_19 |
| 1.43 | Amylase_20 |
| 1.68 | Amylase_21 |
| 1.44 | Amylase_22 |
| 1.39 | Amylase_23 |
| 0.50 | Amylase_24 |
| 1.24 | Amylase_25 |
| 1.08 | Amylase_26 |
| 1.02 | Amylase_27 |
| 1.33 | Amylase_28 |
| 0.88 | Amylase_29 |
| 1.14 | Amylase_31 |
| 0.87 | Amylase_32 |
| 1.07 | Amylase_33 |
| 0.66 | Amylase_34 |
| 1.04 | Amylase_35 |
| 1.09 | Amylase_36 |
| 1.39 | Amylase_37 |
| 1.57 | Amylase_38 |
| 1.37 | Amylase_39 |
| 1.05 | Amylase_40 |
| 1.62 | Amylase_41 |
| 1.02 | Amylase_42 |
| 0.67 | Amylase_43 |
| 0.74 | Amylase_44 |
| 0.89 | Amylase_45 |
| 1.15 | Amylase_46 |
| 0.75 | Amylase_47 |
| 0.87 | Amylase_48 |
| 1.02 | Amylase_49 |
| 1.11 | Amylase_50 |
| 1.06 | Amylase_51 |

### Example 6: Enzyme Stability in Detergent Formulations

The stability of the amylase enzymes in detergent was observed by mixing the enzymes of interest into a defined laundry formulation (Maranil DBS/LC (LAS) 5,5% w/w, Edenor coco fatty acid C12-C18 2.4% w/w, Lutensol AO7 (AEO) 5,5% w/w, Texapon N70 (AES) 5,5% w/w, 1,2 propylene glycol 6,0% w/w, ethanol 2,0% w/w, NaOH 2,2% w/w, 3% Sodium citrate, 150 ppm CaCl2, adjusted to pH 8). The detergents containing enzyme were then stored at 37° C. After several days of storage an aliquot was taken from the detergents containing enzyme and measured for residual activity.

The residual amylase activity was measured by analyzing the rate of p-nitrophenol (pNP) released during the enzymatic hydrolysis of Infinity™ Amylase Liquid Stable Reagent. The sample was diluted in 50 mM MOPS, pH 7.0 prior to the assay. 100 μL of the diluted sample was added to 100 μL of Infinity™ Amylase Liquid Stable Reagent. The reaction was observed at room temperature using a BioTek plate reader at 405 nm. The activity remaining was calculated by comparing the MaxV at each time point to the initial value at day 0.

The results, as provided in the table below, show that the amylases (a variant of Amylase_1 (SEQ ID NO: 146), a variant of Amylase_1 (SEQ ID NO: 148), a variant of Amylase_1 (SEQ ID NO: 152), a variant of Amylase_1 (SEQ ID NO: 170) and Amylase_8 (SEQ ID NO: 15), and a variant of Amylase_8 (SEQ ID NO: 171)) are more stable (have a higher % activity remaining) in detergent formulations over time when compared to the parent enzyme Amylase_51 (SEQ ID NO: 101).

Enzyme stability (% activity remaining)
in defined laundry formulations over time

| 2 Days | 6 Days | Enzyme Name |
|---|---|---|
| 95% | 89% | Amylase_51 (Seq ID 101) |
| 97% | 99% | Amylase_1 (Seq ID 1) |
| 95% | 95% | variant of Amylase_1 (Seq ID 146) |
| 97% | 97% | variant of Amylase_1 (Seq ID 148) |
| 96% | 94% | variant of Amylase_1 (Seq ID 152) |
| 98% | 97% | variant of Amylase_1 (Seq ID 170) |
| 98% | 97% | Amylase_08 (Seq ID 15) |
| 98% | 98% | variant of Amylase_08 (Seq ID 171) |

Additionally, the same set of sequences were tested in Persil Non-Bio. The enzymes were mixed into the detergents and stored at 37° C. After 0 h, 24, and 48 hours an aliquot was taken from the detergents containing enzyme and measured for activity.

The residual amylase activity was measured by analyzing the rate of p-nitrophenol (pNP) released during the enzymatic hydrolysis of Infinity™ Amylase Liquid Stable Reagent. The sample was diluted in 50 mM MOPS, pH 7.0 prior to the assay. 100 μL of the diluted sample was added to 100 μL of Infinity™ Amylase Liquid Stable Reagent. The reaction was observed at room temperature using a BioTek plate reader at 405 nm. The activity remaining was calculated by comparing the MaxV at each time point to the initial value at day 0.

The results, as provided in the table below, show that the amylases (a variant of Amylase_1 (SEQ ID NO: 146), a variant of Amylase_1 (SEQ ID NO: 148), a variant of Amylase_1 (SEQ ID NO: 152), a variant of Amylase_1 (SED IQ NO: 170), Amylase_8 (SEQ ID NO: 15), and a variant of Amylase_8 (SEQ ID NO: 171)) are more stable (have a higher % activity remaining) in detergent formulations over time when compared to the parent enzyme Amylase_51 (SEQ ID NO: 101).

Enzyme stability (% activity remaining)
in detergent formulations over time

| 0 hours | 24 hours | 48 hours | Enzyme Name |
|---|---|---|---|
| 100% | 75% | 53% | Amylase_51 (Seq ID 101) |
| 100% | 100% | 98% | Amylase_1 (Seq ID 1) |
| 100% | 82% | 83% | variant of Amy_1 (Seq ID 146) |
| 100% | 95% | 94% | variant of Amylase_1 (Seq ID 148) |
| 100% | 88% | 87% | variant of Amylase_1 (Seq ID 152) |
| 100% | 92% | 91% | variant of Amylase_1 (Seq ID 170) |
| 100% | 103% | 102% | Amylase_08 (Seq ID 15) |
| 100% | 99% | 100% | variant of Amylase_08 (Seq ID 171) |

### Example 7: Enzyme Performance in Laundry Applications (Stain Removal)

Wash performance was measured for the different concentrations of the hybrid amylases on the stain types CS-28 (colored rice starch) and CS-127 (Potato starch).

Amylases were dosed at 0,004 ppm and 0.02 ppm in a wash solution of 2.7 mM hard water plus 5 g/L model detergent (Maranil DBS/LC (LAS) 5,5% w/w, Edenor coco fatty acid C12-C18 2.4% w/w, Lutensol AO7 (AEO) 5,5% w/w, Texapon N70 (AES) 5,5% w/w, 1,2 propylene glycol 6,0% w/w, ethanol 2,0% w/w, NaOH 2,2% w/w, 3% Sodium citrate, 150 ppm CaCl2, adjusted to pH 8). Wash was conducted in 500 mL wash solution in launder-O-meter for 40 minutes at 40° C. Hereafter, the wash liquor was removed and the fabric was rinsed three times with water. The samples were dried overnight at room temperature. Wash performance was measured using digital image analysis of washed stains. The average RGB intensity was normalized to the parent for each sample or to the value in absence of any amylolytic activity.

The results, as provided in the table below, show that the amylase enzymes being more stable than the parent Amylase_51 have at least similar or improved performance (starch removal) when compared to the parent enzyme (Amylase_51).

| | CS28 | CS28 |
|---|---|---|
| | 0.004 | 0.02 |
| | ppm (in | ppm (in |
| | reference | reference |
| | to control | to control |
| Wash performance results | Amylase_51) | Amylase_51) |
| Amylase_51 (SEQ ID NO: 101) | 100% | 100% |
| Amylase_1 (SEQ ID NO: 1) | 102% | 101% |
| variant of Amylase_1 (SEQ ID NO: 146) | 108% | 102% |
| variant of Amylase_51 (SEQ ID NO: 154) | 105% | 102% |
| variant of Amylase_08 (SEQ ID NO: 171) | 113% | 102% |
| variant of Amylase_1 (SEQ ID NO: 148) | 109% | 103% |
| variant of Amylase_1 (SEQ ID NO: 152) | 110% | 103% |
| Amylase_08 (SEQ ID NO: 15) | 102% | 101% |
| variant of Amylase_1 (SEQ ID NO: 170) | 104% | 103% |

| | CS-127 | CS-127 |
|---|---|---|
| | 0.004 | 0.02 ppm |
| | ppm (in | (in reference |
| | reference | to |
| | to control | control |
| Wash performance results | Amylase_51) | Amylase_51) |
| Amylase_51 (SEQ ID NO: 101) | 100% | 100% |
| Amylase_1 (SEQ ID NO: 1) | 102% | 103% |
| variant of Amylase_1 (SEQ ID NO: 146) | 102% | 105% |
| variant of Amylase_51 (SEQ ID NO: 154) | 102% | 105% |
| variant of Amylase_08 (S SEQ ID NO: 171) | 100% | 102% |
| variant of Amylase_1 (SEQ ID NO: 148) | 103% | 105% |
| variant of Amylase_1 (SEQ ID NO: 152) | 103% | 105% |
| Amylase_08 (SEQ ID NO: 15) | 99% | 102% |
| variant of Amylase_1 (SEQ ID NO: 170) | 106% | 106% |

1. An amylase comprising an amino acid sequence that is at least 70% identical to the amino acid sequence of SEQ ID NO: 133 and comprising at least one amino acid substitution at an amino acid position selected from the group consisting of 25, 26, 27, 28, 30, 32, 35, 36, 37, 113, 242, 245, 246, 250, 251, 254, 268, 269, 271, 272, 273, 275, 375, 376, 377, and 378 according to the numbering of SEQ ID NO: 133.

2. The amylase according to claim 1, wherein the at least one amino acid substitution is at an amino acid position selected from the group consisting of 113, 242, 245, 246, 268, 269, 271, 272, 273, and 275 according to the numbering of SEQ ID NO: 133.

3. The amylase according to claim 1 or 2, wherein the at least one amino acid substitution is selected from the group consisting of G113X, Y242X, F245X, P246X, S268X, Y269X, V271X, N272X, K273X, and H275X according to the numbering of SEQ ID NO: 133.

4. The amylase according to claim 1, wherein the at least one amino acid substitution is at an amino acid position selected from the group consisting of 25, 26, 27, 28, 30, 32, 35, 36, and 37 according to the numbering of SEQ ID NO: 133 and compared to the parent amylase one or more amino acid residue at amino acid position 250, 251, and 254 according to the numbering of SEQ ID NO: 133 is not changed.

5. The amylase according to claim 1, wherein the at least one amino acid substitution is selected from the group consisting of T25X, K26X, V27X, K28X, E30X, A32X, S35X, S36X, and L37X according to the numbering of SEQ ID NO: 133 wherein the amino acid sequence of the amylase comprises one or more of the amino acid residues selected from the group consisting of T250, Y251, and N254 according to the numbering of SEQ ID NO: 133.

6. The amylase according to claim 1, wherein the at least one amino acid substitution is at an amino acid position selected from the group consisting of 250, 251, and 254 according to the numbering of SEQ ID NO: 133 and compared to the parent amylase one or more amino acid residue at amino acid position 25, 26, 27, 28, 30, 32, 35, 36, and 37 according to the numbering of SEQ ID NO: 133 is not changed.

7. The amylase according to claim 1, wherein the at least one amino acid substitution selected from the group consisting of T250X, Y251X, and N254X according to the numbering of SEQ ID NO: 133 and wherein the amino acid sequence of the amylase comprises one or more of the amino acid residues selected from the group consisting of T25, K26, V27, K28, E30, A32, S35, S36, and L37 according to the numbering of SEQ ID NO: 133.

8. The amylase according to claim 1, wherein the at least one amino acid substitution is at an amino acid position selected from the group consisting of 25, 26, 27, 28, 30, 32, 35, 36, and 37 according to the numbering of SEQ ID NO: 133 and compared to the parent amylase one or more amino acid residue at amino acid position 375, 376, 377, and 378 according to the numbering of SEQ ID NO: 133 is not changed.

9. The amylase according to claim 1, wherein the at least one amino acid substitution is selected from the group consisting of T25X, K26X, V27X, K28X, E30X, A32X, S35X, S36X, and L37X according to the numbering of SEQ ID NO: 133 and wherein the amino acid sequence of the amylase comprises one or more of the amino acid residues selected from the group consisting of K375, Y376, N377, and I378 according to the numbering of SEQ ID NO: 133.

10. The amylase according to claim 1, wherein the at least one amino acid substitution is at an amino acid position selected from the group consisting of 375, 376, 377, and 378 according to the numbering of SEQ ID NO: 133 and compared to the parent amylase one or more amino acid residue at amino acid position 25, 26, 27, 28, 30, 32, 35, 36, and 37 according to the numbering of SEQ ID NO: 133 is not changed.

11. The amylase according to claim 1, wherein the at least one amino acid substitution is selected from the group consisting of K375X, Y376X, N377X, and I378X according to the numbering of SEQ ID NO: 133 and wherein the amino acid sequence of the amylase comprises one or more of the amino acid residues selected from the group consisting of T25, K26, V27, K28, E30, A32, S35, S36, and L37 according to the numbering of SEQ ID NO: 133.

12. The amylase according to claim 1, wherein the amino acid sequence of the amylase comprises one or more deletions of amino acid residues at positions selected from the group consisting of 180, 181, 182, and 183 according to the numbering of SEQ ID NO: 133.

13. The amylase according to claim 1, wherein the amino acid sequence of the amylase comprises a deletion of the C-terminus.

14. The amylase according to claim 1, wherein the amino acid sequence of the amylase comprises at least one amino acid substitution at a position selected from the group consisting of 87, 98, 109, 130, 149, 178, 185, 189, 194, 201, 225, 242, 243, 272, 282, 309, 320, 359, 439, and 444 according to the numbering of SEQ ID NO: 133.

15. The amylase according to claim 1, wherein the amino acid sequence of the amylase comprises at least one region selected from the regions consisting of region 1, region 2, region 3, and region 4;
wherein region 1 is an amino acid sequence having at least 75% identity to the amino acid sequence of SEQ ID NO:103, or SEQ ID NO:104;
wherein region 2 is an amino acid sequence having at least 75% identity to an amino acid sequence selected from: SEQ ID NO:105, SEQ ID NO:106, SEQ ID NO:107, SEQ ID NO:108, SEQ ID NO:109, SEQ ID NO:110, SEQ ID NO:111, and SEQ ID NO:112;
wherein region 3 is an amino acid sequence having at least 75% identity to an amino acid sequence selected from: SEQ ID NO:113, SEQ ID NO:114, SEQ ID NO:115, SEQ ID NO:116, SEQ ID NO:117, SEQ ID NO:118, SEQ ID NO:119, and SEQ ID NO:120; and
wherein region 4 is an amino acid sequence having at least 75% identity to an amino acid sequence selected from SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, and SEQ ID NO:130.

16. The amylase according to claim 1, comprising an amino acid sequence that is at least 80% identical to the amino acid sequence of: SEQ ID NO:1, SEQ ID NO:3, SEQ ID NO:5, SEQ ID NO:7, SEQ ID NO:9, SEQ ID NO:11, SEQ ID NO:12, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, or SEQ ID NO:171.

17. The amylase according to claim 1, wherein the amylase has an increase in expression; activity; thermostability; stability; performance in laundry; or any combination thereof.

18. A composition comprising the amylase as in claim 1.

19. The composition of claim 18, further comprising at least one second enzyme selected from the group consisting of: a second amylase, a lipase, a protease, a cellulase, a laccase, a mannanase, a pectinase, and a nuclease.

20. A method of making the amylase of claim 1, comprising: providing a nucleic acid sequence encoding the amylase, transforming the nucleic acid sequence into an expression host, cultivating the expression host to produce the amylase, and purifying the amylase.

21. A method of preparing a dough or a baked product prepared from the dough, the method comprising adding the amylase of claim 1 to the dough and baking it.

22. (canceled)
